(19) United States
(12) Patent Application Publication
(10) Pub. No.: US 2005/0136039 A1
(43) Pub. Date: Jun. 23, 2005

(54) Adipocytes and uses thereof

(75) Inventor: Ronald Kahn, West Newton, MA (US)

Correspondence Address: FISH & RICHARDSON PC, 225 FRANKLIN ST, BOSTON, MA, 02110, US

(73) Assignee: Joslin Diabetes Center, Inc., Boston, MA (US)

(21) Appl. No.: 10/946,843

(22) Filed: Sep. 22, 2004

Related U.S. Application Data
(63) Continuation of application No. PCT/US03/08978, filed on Mar. 24, 2003.
(60) Provisional application No. 60/366,800, filed on Mar. 22, 2002.

(51) Int. Cl. A61K 45/00; C12N 5/08

(52) U.S. Cl. 424/93.7; 435/366

(57) ABSTRACT

The invention features methods of making and using subpopulations or subtypes of adipose cells based on their differential regulation of gene expression, size or function in response to modulation of insulin signaling.

### CROSS REFERENCE TO RELATED APPLICATIONS

This application is a continuation of International Patent Application No. PCT/US03/08978, filed Mar. 24, 2003, which claims the benefit of U.S. Provisional Application 60/366,800, filed Mar. 22, 2002, the entire contents of which are hereby incorporated by referenced.

### BACKGROUND OF THE INVENTION

Type 2 diabetes is characterized by insulin resistance in muscle, liver and fat and by defects in insulin secretion from the pancreatic β cell (Martin et al., 1992; Kahn, 1994). Muscle-specific insulin receptor knockout mice do not show major defects in glucose metabolism (Brining et al., 1998), whereas β cell-specific insulin receptor knockout mice have impaired glucose tolerance due to a selective loss of first phase glucose-stimulated insulin secretion (Kulkarni et al., 1999). Liver-specific insulin receptor knockout mice exhibit insulin resistance, moderate glucose intolerance and a failure of insulin to suppress hepatic glucose production and to regulate hepatic gene expression (Michael et al., 2000).

The role of white adipose tissue in overall glucose homeostasis is not clear. Although some studies suggest that adipose tissue in humans may metabolize up to 20% of an orally-administered glucose load (Jansson et al., 1994; Kashiwagi et al., 1983), euglycemic hyperinsulinemic clamp studies in rats indicate that adipose tissue is responsible for only 3-5% of glucose storage (James et al., 1985). On the other hand, adipose selective inactivation of the GLUT4 gene causes glucose intolerance and hyperinsulinemia, and induces secondary alterations in insulin action in muscle and liver (Abel et al., 2001).

### SUMMARY OF THE INVENTION

The invention is based, in part, on the inventor's discovery fat-specific, e.g., adipose tissue-specific, e.g., white adipose tissue (WAT)-specific, reduction of insulin receptor signaling (e.g., disruption of the insulin receptor) in an animal causes one or more of: (a) a decrease in fat mass and whole body triglyceride stores, (b) loss of the normal relationship between plasma leptin and body weight, (c) protection against obesity, e.g., obesity related to aging and overeating, and obesity-related glucose intolerance, (d) increased longevity; and (e) unmasking of a heterogeneity, e.g., a heterogeneity in gene expression, size and/or function, in WAT cells, e.g., within a single fat depot or among different depots (fore example, visceral vs. peripheral fat depots).

Accordingly, in one aspect, the invention features a fat cell or population of fat cells, e.g., an isolated population of white adipose tissue (WAT) cell. The cell or isolated population can exhibit modulated expression, e.g., increased or decreased expression, of at least one gene, e.g., in response to modulated insulin signaling. The gene is preferably one or more of: fatty acid synthase (FAS), SREBP-1c, C/EBPα, β3 adrenergic receptor, fatty acid transport protein 1 (FATP 1), fatty acid transport protein aP2 (FATP aP2), carnitine palmitoyltransferase 2, non-muscle myosin form A, acetyl CoA-dehydrogenase, citrate synthase, cytochrome C, vimentin, EH domain containing protein 2, elongation factor 2, glucose regulated protein 78 (GRP 78), transketolase, succinyl CoA transferase, activating transcription factor 3, TGFβ, PAI 1, annexin 2, annexin A6, 47 kDa heat shock protein, PDGF receptor, lumican and colony stimulating factor 3 (CSF3). Modulation of the gene is preferably evaluated compared to a reference, e.g., a control value. For example, modulation of the gene can be compared to (a) the expression of the same gene in the adipose tissue from which the isolated population is derived; (b) the expression of the same gene in a different population of adipocytes that does not exhibit modulation in expression of the gene in response to modulation of insulin signaling; or (c) the expression of the same gene in the absence of modulated insulin signaling.

In a preferred embodiment, the population exhibits decreased expression, levels or activity of one or more of: FAS, SREBP-1c, C/EBPα, β3 adrenergic receptor, FATP1, FATP aP2, carnitine palmitoyltransferase 2, non-muscle myosin form A, acetyl CoA-dehydrogenase, citrate synthase and cytochrome C, e.g., in response to inhibition of insulin signaling, e.g., in response to a reduction in expression or activity of IR, insulin receptor substrate (IRS), phosphatidylinositol 3-kinase (PI3K), Akt, PKC, SHC, SHP-2, GRB2, SOS-1or Ras, but preferably inhibition of IR.

In a preferred embodiment, the population has (1) increased levels of adiponectin and/or ACRP30 compared to the expression of the same gene in the absence of modulated insulin signaling and/or (2) unchanged levels of any of IRS-1, IRS-2, GLUT-4, PPARγ, leptin, and aP2 compared to the control.

In a preferred embodiment, the cells of the isolated population have an average diameter that is at least 20% smaller (preferably at least 25%, 30%, 40% or 50% smaller) than the average diameter of the fat cells, e.g., WAT cells, from which the isolated population is derived (e.g., the in vivo or ex vivo fat tissue from which the isolated population is derived). In a preferred embodiment, the cells of the isolated population have an average diameter smaller than 100 μm, preferably smaller than 75 μm, more preferably smaller than 50 μm or smaller than 25 μm.

In a preferred embodiment, the population has decreased basal lipogenesis activity and/or decreased isoproterenol induced lipolysis.

In another aspect, the invention features an isolated cell or population of white adipose tissue (WAT) cells having an average diameter smaller than 80% of the average diameter of the WAT cells before isolation. “Before isolation” means that the WAT cells have not been dissociated from other tissue components in the tissue (e.g., the fat tissue) from which they derive. A WAT cell “before isolation” includes, e.g., a WAT cell within the body of an animal, or a WAT cell in a fat tissue that has been removed from the body of an animal, but has not yet been dissociated, e.g., by enzymatic digestion (e.g., collagenase digestion).

In a preferred embodiment, the cells of the isolated population have an average diameter smaller than 100 μm, preferably smaller than 90 μm, 80 μm, or 75 μm, more preferably smaller than 50 μm or smaller than 25 μm.

In a preferred embodiment, the population has modulated gene expression, e.g., decreased transcription of one or more of: fatty acid synthase (FAS), SREBP-1c, C/EBPα, β3 adrenergic receptor, and fatty acid transport protein 1 (FATP1), in response to inhibition of insulin signaling. The population can also have decreased levels or activity of one or more of: fatty acid synthase (FAS), fatty acid transport protein aP2 (FATP aP2), carnitine palmitoyltransferase 2, non-muscle myosin form A, acetyl CoA-dehydrogenase, citrate synthase and cytochrome C.

In a preferred embodiment, the population has (1) increased levels of adiponectin and/or ACRP30 compared to the expression of the same gene in the absence of modulated insulin signaling and/or (2) unchanged levels of any of IRS-1, IRS-2, GLUT-4, PPARγ, leptin, and aP2 compared to the control.

In a preferred embodiment, the population has decreased basal lipogenesis activity and/or isoproterenol induced lipolysis activity.

The isolated population can be cultured, e.g., in vitro.

In another aspect, the invention features an isolated WAT cell or a population of isolated WAT cells which, in response to inhibition of insulin signaling, exhibit an average diameter of at least 120% of the median diameter of the white adipose cells before isolation. Preferably, the population has an average diameter of at least 100 μm, more preferably at least 125 μm, more preferably at least 150 μm.

In a preferred embodiment, the cells have increased basal lipogenesis and/or increased basal lipolysis activity.

In another aspect, the invention features a method of evaluating a cell, e.g., identifying or selecting an adipocyte subtype or subpopulation of adipocytes that exhibits modulation of gene expression compared to other adipocytes, e.g., in response to modulation, e.g., inhibition, of insulin signaling, e.g., a population of cells described herein. The method includes: providing a test adipocyte; and evaluating expression, level or activity of one or more of: fatty acid synthase (FAS), SREBP-1c, C/EBPα, β3 adrenergic receptor, fatty acid transport protein 1 (FATP1) fatty acid transport protein aP2 (FATP aP2), carnitine palmitoyltransferase 2, non-muscle myosin form A, acetyl CoA-dehydrogenase, citrate synthase and cytochrome C in the test adipocyte. The test adipocyte is identified as the desired adipocyte subtype or selected if the gene expression or level or activity is decreased compared to a reference.

In a preferred embodiment, the method also includes a step of isolating the selected cell.

In a preferred embodiment, the method also includes a step of inhibiting insulin signaling in the cell, e.g., inhibiting an insulin receptor or another molecule involved in insulin signal described herein.

In some embodiments, the method also includes evaluating an adipocyte function, e.g., glucose uptake, lipogenesis or lipolysis, in the identified cell.

In a preferred embodiment, the method also includes evaluating the expression or activity of a cell surface marker in the adipocyte, e.g., of one or more of CD13, CD29, CD34, CD44, CD49, CD49d, CD54, CD58, CD90, CD105, VEGF-R, and SH-3. A cell surface marker of the adipocyte can be correlated with a characteristic of the adipocyte population described herein.

In another aspect, the invention features a cell or tissue, e.g., an isolated cell or tissue, e.g., an isolated adipose cell or tissue, e.g., an isolated WAT cell, in which insulin receptor signaling is disrupted. In a preferred embodiment, the cell has been administered an agent that inhibits a component of the insulin receptor signaling pathway, e.g., an agent that inhibits a component of the insulin receptor signaling pathway described herein.

In a preferred embodiment, the cell is an isolated adipocyte, e.g., a WAT adipocyte.

In a preferred embodiment, the activity, level or gene expression of IR in the cell is reduced.

In a preferred embodiment, the adipocyte is a genetically engineered cell having a disruption in a gene encoding a component of the insulin receptor signaling pathway, e.g., insulin, IR, IRS, P13K, Akt, PKC, SHC, SHP-2, GRB2, SOS-1 or Ras. In a preferred embodiment, the IR gene is disrupted, e.g., the IR gene is knocked-out.

The cell can be implanted into a subject, e.g., a human or non-human animal. The cell implanted into the subject can be autologous, allogeneic, or xenogeneic.

In another aspect, the invention features a method of evaluating a treatment or compound, e.g., for its effect on fat cell development, function, or size. The method includes: providing or obtaining a WAT cell or cell population described herein, e.g., a population that exhibits decreased expression, levels or activity of one or more of: FAS, SREBP-1c, C/EBPα, β3 adrenergic receptor, FATP1, FATP aP2, carnitine palmitoyltransferase 2, non-muscle myosin form A, acetyl CoA-dehydrogenase, citrate synthase and cytochrome C in response to inhibition of insulin signaling; contacting a test treatment or compound with the cell or cell population; and evaluating a parameter of fat cell development, function, or size. The method is useful, e.g., in drug discovery, drug characterization, or drug quality control methods.

The evaluation can include any one or more of:
(a) evaluating gene expression in the cell or cell population (e.g., evaluating expression, levels or activity of FAS, SREBP-1c, C/EBPα, β3 adrenergic receptor, FATP1, FATP aP2, carnitine palmitoyltransferase 2, non-muscle myosin form A, acetyl CoA-dehydrogenase, citrate synthase, cytochrome C, vimentin, EH domain containing protein 2, elongation factor 2, GRP 78, transketolase, succinyl CoA transferase, activating transcription factor 3, TGFβ, PAI 1, annexin 2, annexin A6, 47 kDa heat shock protein, PDGF receptor, lumican or CSF3);
(b) evaluating glucose uptake, lipogenesis and/or lipolysis;
(c) evaluating expression of one or more of CD13, CD29, CD34, CD44, CD49, CD49d, CD54, CD58, CD90, CD105, VEGF-R, and SH-3;
(d) evaluating cell size, e.g. diameter, e.g., average cell diameter.

The evaluation step can be performed once or more than once, e.g., at least two times. In some embodiments, two evaluations are performed, e.g., two evaluations separated by at least a day, a week, a month or more.

The test treatment or compound can be a test drug for obesity or an obesity related disorder, e.g., diabetes. The test treatment can be, e.g., a small molecule, a polypeptide or polypeptide mimetic, or a nucleic acid. In another preferred embodiment, the test agent is a member of a combinatorial library, e.g., a peptide or organic combinatorial library, or a natural product library. In a preferred embodiment, a plurality of test agents, e.g., library members, is tested. Preferably, the test agents of the plurality, e.g., library, share structural or functional characteristics.

In a preferred embodiment, the method also includes administering the test treatment or compound to a test animal, e.g., an animal model of diabetes or obesity. In some embodiment, the animal is a transgenic animal, e.g., a transgenic animal having an adipocyte-specific knock-out or overexpressing mutation for a component of the insulin receptor signaling pathway, e.g., a FIRKO mouse described herein.

In another aspect, the invention features a method of evaluating a subject, e.g., evaluating a subject's predisposition or risk of obesity or an obesity related disorder. The method includes evaluating one or more cells of the subject's adipose tissue, e.g., evaluating the presence or level of an adipocyte subtype described herein in the subject.

In a preferred embodiment, the method includes evaluating the subject for the presence or amount of TG-S adipocytes and/or TG-L adipocytes, e.g., in a fat tissue of the subject. TG-S and TG-L adipocytes are described herein in the detailed description and can be identified by routine techniques by any of: gene expression, size or function, e.g., in response to reduced or inhibited insulin signaling, e.g., a gene expression, size or function of a TG-L or TG-S adipocyte described herein.

In a preferred embodiment, a fat tissue sample or an adipose cell sample (e.g., from subcutaneous or visceral fat tissue, but preferably subcutaneous) is obtained from the subject and the sample is evaluated for the presence or amount of TG-S and/or TG-L adipocytes. In one embodiment, a ratio of TG-S to TG-L adipocytes in the sample is determined. In one embodiment, the sample is contacted with an agent that decreases or inhibits insulin signaling (e.g., an agent, e.g., an antisense nucleic acid or antibody that inhibits IR); and the cells of the sample are assayed for gene expression, lipolysis, lipogenesis, or glucose uptake.

In one embodiment, an increased number or ratio of TG-L cells in the subject (e.g., compared to a reference value) is correlated with a higher predisposition or risk of obesity or an obesity related disorder. An increased number or ratio of TG-S cells in the subject (e.g., compared to a reference value) is correlated with a lower predisposition or risk of obesity or an obesity related disorder. For example, a ratio of TG-S to TG-L greater than about 1:1 can be correlated with a lower predisposition or risk of obesity or an obesity related disorder.

In one embodiment, the result of the evaluation is used to identify or select a treatment for the subject.

In one embodiment, the method also includes evaluating the subject for one or more of: insulin metabolism, weight, glucose levels, diet.

In another aspect, the invention features a method of modulating an adipocyte, e.g., modulating differentiation, size or function of the adipocyte.

In one embodiment, the method includes: providing, obtaining or identifying an adipocyte; and modulating in the adipocyte the expression, levels or activity of a gene or protein that is differentially regulated by insulin in adipose tissue, e.g., one or more of: fatty acid synthase (FAS), SREBP-1c, C/EBPα, β3 adrenergic receptor, fatty acid transport protein 1 (FATP 1), fatty acid transport protein aP2 (FATP aP2), carnitine palmitoyltransferase 2, non-muscle myosin form A, acetyl CoA-dehydrogenase, citrate synthase, cytochrome C, vimentin, EH domain containing protein 2, elongation factor 2, glucose regulated protein 78 (GRP 78), transketolase, succinyl CoA transferase, activating transcription factor 3, TGFβ, PAI 1, annexin 2, annexin A6, 47 kDa heat shock protein, PDGF receptor, lumican, and colony stimulating factor 3.

In a preferred embodiment, the expression, levels or activity is modulated in vitro. In some embodiments, the expression, levels or activity is modulated in vitro and the modulated adipocyte is transplanted into a subject. In other embodiments, the expression, levels or activity is modulated in vivo.

In a preferred embodiment, the method includes administering to the subject an agent that modulates, e.g., decreases, the expression, levels or activity of the gene or protein. In a preferred embodiment, the agent is specifically targeted to the fat tissue, e.g., WAT tissue, of the subject.

In a preferred embodiment, at least one of (preferably at least two of, more preferably at least three or more of) FAS, SREBP-1c, C/EBPα, β3 adrenergic receptor, and FATP 1 expression or activity is decreased.

In a preferred embodiment, the method includes modulating a TG-L adipocyte.

In a preferred embodiment, the adipocyte is from a subject with unwanted weight or fat, e.g., an obese subject.

In a preferred embodiment, the method includes inhibiting insulin signaling in the adipocyte.

In another aspect, the invention features a method of evaluating a subject, e.g., identifying a subject, having or at risk for diabetes or a diabetes related condition (e.g., atherosclerosis, dyslipidemia, hypertension). The method includes evaluating a fat cell or tissue of the subject for a marker, e.g., an intracellular or cell surface marker, of a visceral fat depot. The marker can be the expression, level, or activity of one or more of: cytochrome p450 1b1, 17-beta-estradiol, angiotensinogen, thrombospondin 1, mevalonate kinase, and prolactin receptor. An abnormal or aberrant level of the marker compared to a reference, is correlated with presence or risk of diabetes or a diabetes related condition. The reference can be, e.g., a control value, a value for a non-visceral fat cell or tissue, or a predetermined level, e.g., a basal level.

In a preferred embodiment, a reduced level of one or more of: cytochrome p450 1b1, 17-beta-estradiol, angiotensinogen, thrombospondin 1, mevalonate kinase, and prolactin receptor, compared to a reference, is correlated with presence or risk for diabetes or a diabetes related condition.

The fat cell or tissue is preferably (or preferably derived from) a visceral fat depot. The subject can be a non-human experimental animal, domestic animal, e.g., food animal, or a human.

In a preferred embodiment, the method includes providing a record, e.g., a print or computer readable material, e.g., an informational, diagnostic, or instructional material, e.g., to the subject, health care provider, or an insurance company, identifying the abnormal or aberrant marker as a risk or diagnostic factor for diabetes or a related condition.

In a preferred embodiment, the method includes detecting a genetic lesion or mutation in the marker. The sequence of human cytochrome p450 1b1 is known and can be found, e.g., in Sutter, J. Biol. Chem. 269 (18), 13092-13099 (1994). The sequence of angiotensinogen is known and can be found, e.g., in Gaillard, DNA 8 (2), 87-99 (1989). The sequence of TSP-1 is known and can be found at: Hennessy, J. Cell Biol. 108 (2), 729-736 (1989). The sequence of mevalonate kinase can be found at Schafer, J. Biol. Chem. 267 (19), 13229-13238 (1992). The sequence of prolactin receptor can be found at Hu et al., J. Clin. Endocrinol. Metab. 84 (3), 1153-1156 (1999).

In a preferred embodiment, the method includes evaluating the level of expression of the marker gene, e.g., evaluating the amount or half life of the marker mRNA. Over- or under-expression of a marker gene, compared to a control, can be evaluated by, e.g., Northern blot, TaqMan assay, or other methods known in the art.

In a preferred embodiment, the method includes evaluating a marker activity, e.g., enzymatic activity or substrate binding activity.

In a preferred embodiment, the method includes evaluating the presence or level of the marker protein.

In a preferred embodiment, the method includes treating the subject for the diabetes or related condition.

In a preferred embodiment, the subject is further evaluated for one or more of the following parameters: (1) insulin level (2) weight; (3) glucose levels.

In a preferred embodiment, the evaluation is used to choose a course of treatment.

Methods of the invention can be used prenatally or to determine if a subject's offspring will be at risk for a disorder.

In a preferred embodiment, the fat cell or tissue is evaluated in vitro. In other embodiments, the fat cell or tissue is evaluated in vivo.

In another aspect, the invention features a method of evaluating a subject, e.g., identifying a subject having, or at risk for, diabetes or a diabetes related condition (atherosclerosis, dyslipidemia, hypertension). The method includes evaluating a fat cell or tissue of the subject for a marker, e.g., an intracellular or cell surface marker, of a specific fat depot, e.g., a visceral fat depot. The marker can be the level, expression or activity of one or more mitochondrial protein, e.g., a protein in the mitochondrial oxidative phosphorylation pathway, preferably cytochrome C oxidase subunit VIIIb, cytochrome C oxidase subunit VIIa (Arnaudo et al., Gene 119 (2), 299-305 (1992)), or an uncoupling protein, e.g., UCP1 (Cassard, J. Cell. Biochem. 43 (3), 255-264 (1990); or UCP3 (Vidal-Puig, Biochem. Biophys. Res. Commun. 235 (1), 79-82 (1997)). An abnormal or aberrant level of the marker compared to a reference, is correlated with presence or risk of diabetes or a diabetes related condition. The reference can be a control value, a value for a non-visceral fat cell or tissue, or a predetermined level, e.g., a basal level.

In a preferred embodiment, a reduced level of one or more of: cytochrome C oxidase subunit VIIIb, cytochrome C oxidase subunit VIIa, UCP1 or UCP3, compared to a reference, is correlated with presence or risk for diabetes or a diabetes related condition.

The fat cell or tissue is preferably (or preferably derived from) a visceral fat depot. The subject can be a non-human experimental animal, domestic animal, e.g., food animal, or a human.

In a preferred embodiment, the method includes providing a record, e.g., a print or computer readable material, e.g., an informational, diagnostic, or instructional material, e.g., to the subject, health care provider, or an insurance company, identifying the abnormal or aberrant marker as a risk or diagnostic factor for diabetes or a related condition.

In a preferred embodiment, the method includes detecting a genetic lesion or mutation in the marker.

In a preferred embodiment, the method includes evaluating the level of expression of the marker gene, e.g., evaluating the amount or half life of a mRNA. Over- or under-expression of a gene, compared to a control, can be evaluated by, e.g., Northern blot, TaqMan assay, or other methods known in the art.

In a preferred embodiment, the method includes evaluating a marker activity, e.g., enzymatic activity or substrate binding activity.

In a preferred embodiment, the method includes evaluating the presence or level of the marker protein.

In a preferred embodiment, the method includes treating the subject for the diabetes or related condition.

In a preferred embodiment, the subject is further evaluated for one or more of the following parameters: (1) insulin level (2) weight; (3) glucose levels.

In a preferred embodiment, the evaluation is used to choose a course of treatment.

Methods of the invention can be used prenatally or to determine if a subject's offspring will be at risk for a disorder.

In a preferred embodiment, the fat cell or tissue is evaluated in vitro. In other embodiments, the fat cell or tissue is evaluated in vivo.

In another aspect, the invention features a method of treating a subject, e.g., for diabetes or a diabetes related disorder. The method includes modulating in a fat cell or tissue of the subject one or more marker, e.g., an intracellular or cell surface marker. The marker can be one or more of: cytochrome p450 1b1, 17-beta-estradiol, angiotensinogen, TSP 1, mevalonate kinase, prolactin receptor, and a protein in the mitochondrial oxidative phosphorylation pathway, e.g., cytochrome C oxidase subunit VIIIb, cytochrome C oxidase subunit VIIa, and one or more uncoupling protein, e.g., UCP1 or UCP3.

In a preferred embodiment, the level, activity or level of the marker, e.g., a marker described herein, is increased.

In a preferred embodiment, the method includes administering an agent that modulates, preferably increases, the marker.

In a preferred embodiment, the agent is targeted, e.g., specifically targeted, to a fat tissue, preferably a visceral fat depot.

As used herein, “an isolated population” of adipocytes is a population of adipocytes that is enriched in adipocytes compared to the source of the adipocytes. The source can be a tissue, e.g., fat tissue, taken directly from a subject, e.g., from a liposuction or other surgery procedure. The subject can be an adult, fetal or neonatal subject. Preferably, the isolated population is substantially free of non-adipocyte cells and non-cellular material present in a fat tissue. Non-adipocyte cells normally present in a fat tissue include, e.g., fibroblastic connective tissue cells, leukocytes, macrophages, or pre-adipocytes. Non-cellular material normally present in a fat tissue includes, e.g., collagen fibers. “Substantially free” means that the cell population contains less than 25% non-adipocyte cells and non-cellular material on a weight by weight basis. Preferably, the cell population contains less than 20%, more preferably less than 15%, even more preferably less than 10% or 5% non-adipocyte cells and non-cellular material on a weight by weight basis. An isolated population includes a population of cultured cells derived from an isolated population of adipocyte cells described herein.

As used herein, “treatment” or “treating a subject” is defined as the application or administration of a therapeutic agent to a patient, or application or administration of a therapeutic agent to an isolated tissue or cell line from a patient, who has a disease, a symptom of disease or a predisposition toward a disease. Treatment can slow, cure, heal, alleviate, relieve, alter, remedy, ameliorate, improve or affect the disease, a symptom of the disease or the predisposition toward disease, e.g., by at least 10%.

As used herein, to ability of a first molecule to “interact” with a second molecule refers to the ability of the first molecule to act upon the structure and/or activity of the second molecule, either directly or indirectly. For example, a first molecule can interact with a second by (a) directly binding, e.g., specifically binding, the second molecule, e.g., transiently or stably binding the second molecule; (b) modifying the second molecule, e.g., by cleaving a bond, e.g., a covalent bond, in the second molecule, or adding or removing a chemical group to or from the second molecule, e.g., adding or removing a phosphate group or carbohydrate group; (c) modulating an enzyme that modifies the second molecule, e.g., inhibiting or activating a kinase or phosphatase that normally modifies the second molecule; (d) affecting expression of the second molecule, e.g., by binding, activating, or inhibiting a control region of a gene encoding the second molecule, or binding, activating, or inhibiting a transcription factor that associates with the gene encoding the second molecule; (d) affecting the stability of an mRNA encoding the second molecule, e.g., by inhibiting mRNAse activity against the mRNA encoding the second molecule or by degrading the mRNA encoding the second molecule.

### DESCRIPTION OF THE DRAWINGS

FIG. 1 is a set of diagrams and photographs of the transgene construct, and assessment of insulin receptor recombination and receptor expression. (a) Representation of aP2-Cre transgene. (b) Schematic of the IR lox allele before and after recombination. The position of the different primers used in the PCR analysis is shown by the arrows labeled P1, P2, P3. The knockout allele is shown below the floxed allele, indicating the deletion of exon 4 in the event of recombination of the insulin receptor gene. B, BamHI; S, SalI; Sc, Sac1 restriction sites, NLS, nuclear localization signal. (c) Results from PCR analysis of DNA prepared from isolated adipocytes. DNA from isolated adipocytes of FIRKO mice produced a 220 bp band (lane 1) suggesting a recombination event; a 250 bp band was detected in WT mice (lane 2) and a 300 bp band, containing the loxP site, was observed in adipocytes from IR lox mice (lane 3). (d) Western blot analysis of skeletal muscle, heart, liver, brain, brown adipose tissue (BAT) and white adipose tissue (WAT) of eight pooled FIRKO mice.

FIG. 2 is a set of graphs showing glucose uptake in isolated adipocytes, body weight, gonadal fat pad mass and whole body triglyceride stores in FIRKO mice and controls. (a) Dose-response curves for insulin stimulated U-¹⁴C-glucose uptake in isolated adipocytes from 3 month old male FIRKO mice (n=6) and WT, IR 10× and aP2-Cre control littermates (n=16). Values at insulin concentrations of 0.05 nM and higher are significantly different between FIRKO mice and controls (* p<0.05). (b) Body weight, (c) gonadal fat pad mass and (d) whole body triglyceride stores in FIRKO mice and controls [WT, aP2-Cre, and IR (lox/lox)] determined using 4 month-old males. Each bar represents the mean±SEM of 12 animals of each genotype for body weight and fat pad mass and 6 animals for the triglyceride content. ns=not significant; * indicates P<0.05.

FIG. 3 is a set of graphs showing the altered relationship between plasma leptin levels and body weight or gonadal fat pad mass in FIRKO mice. Plasma leptin levels were measured in triplicate using an ELISA assay. Panel (a) shows that FIRKO mice had significantly (p<0.05) higher plasma leptin levels in relation to gonadal fat pad mass compared to control littermates. Data represent the mean±SEM of 15 animals per genotype (*p<0.05). In panel (b), plasma leptin levels are expressed in relation to body weight (g) in 2 month old male FIRKO mice and control littermates. In WT, aP2-Cre, and IR(lox/lox) mice plasma leptin levels correlated with the body weight (r=0.732, p<0.05), whereas leptin levels for the FIRKO mice (filled circles) were not related to body mass. In panel (c), plasma leptin levels at 12 weeks after GTG (male, initial dose at 7 weeks) or saline treatment in FIRKO and control mice are plotted. The increase in plasma leptin levels after GTG induced obesity and hyperphagia (see FIG. 4) in all genotypes was significantly lower in FIRKO mice compared to controls. (* p<0.05). Data represent the mean±SEM of at least 8 animals per genotype.

FIG. 4 is a set of graphs showing the results of glucose tolerance tests. FIRKO mice are protected from age related glucose intolerance and insulin resistance. Panel (a) shows glucose tolerance tests performed on 2-month-old, panel (b) on 10 month old male WT, IR (lox/lox), aP2-Cre, and FIRKO mice as described in Methods. Results are expressed as mean±SEM from at least 8 animals per genotype. Values at 15, 30, 60, and 120 min are significantly different between FIRKO mice and controls (WT, IR (lox/lox), aP2-Cre) (*p<0.05). Panel (c) shows insulin tolerance tests, performed on random-fed, 2 month-old and panel (d) 10 month-old male WT, IR (lox/lox), aP2-Cre, and FIRKO mice as described in Methods. Results are expressed as mean percent of basal blood glucose concentration+SEM for at least eight animals per genotype. Values at 30 and 60 min are significantly different between FIRKO mice and controls (WT, IR (lox/lox), aP2-Cre) (*p<0.05).

FIG. 5 is a set of graphs showing the effect of gold thioglucose (GTG) on FIRKO mice. Male FIRKO mice and controls were given 0.5 mg/g body weight GTG at 6 weeks of age. (a) Food intake was determined daily over a week before and 12 weeks after GTG injection. Data represent the mean±SEM of at least 8 animals per genotype. The daily food intake increased by ˜125% in FIRKO and control littermates after GTG treatment (p<0.05). Panel (b) shows the body weight gain 12 weeks after GTG (male, initial dose at 6 weeks) or saline treatment in FIRKO and control mice. There was no significant difference in the initial weight at 4 weeks between all genotypes. Despite the increased food intake after GTG treatment, FIRKO mice were protected from the increase in body weight in GTG treated controls compared to the saline group (* P<0.05). (c) Glucose tolerance tests, 12 weeks after GTG-induced obesity in FIRKO mice and control littermates. Values at all time points were significantly different between FIRKO mice and controls (WT, IR (lox/lox), aP2-Cre) (*p<0.05). (d) Insulin tolerance tests, 12 weeks after GTG-induced obesity in FIRKO mice and control littermates. Values at 30 min and 60 min were significantly different between FIRKO mice and controls (WT, IR (lox/lox), aP2-Cre) (*p<0.05).

FIG. 6 is a set of photographs and graphs illustrating the heterogeneity of adipose tissue in FIRKO mice. Tissue of FIRKO mice displays heterogeneity in cell size and impairment of insulin stimulated glucose uptake. (a) Hematoxylin and eosin staining of white adipose tissue sections from random-fed, 4 month-old male FIRKO and WT mice. Initial magnification, 40×. (b) The distribution curve of diameter for 100 measured fat cells per slide shows a bimodal distribution in adipocytes of FIRKO mice with two peaks (small adipocytes, diameter 25-75 μm and large adipocytes, diameter 100-150 μm). (c) The diameter distribution curve for controls showed a normal distribution. Data represent the mean±SEM of 10 slides from six mice. Data represent the mean±SEM of 10 slides from six mice. (d) Basal and insulin stimulated glucose uptake in adipocytes from 3 month old male FIRKO mice was not different in any cell size range confirming the knockout of the insulin receptor in the FIRKO mice. Adipocytes from epigonadal fat pads of 4 WT and 8 FIRKO mice were isolated, pooled and then separated into different diameter ranges as described in Methods. Insulin stimulation was performed for 30 min at 100 nM. Data represent the mean±SEM of 5 independent experiments. (e) Basal and insulin-stimulated glucose uptake in adipocytes from 3 month-old male WT mice. Basal glucose uptake was significantly lower in the adipocytes of a diameter >150 μm, but not different between the other cell size fractions. Adipocytes of a diameter <100 μm had significantly higher glucose uptake after insulin stimulation compared to adipocytes of a diameter >100 μm.

FIG. 7 is a set of graphs and blots illustrating differences in gene expression as a function of size in wild type vs. FIRKO mice. Differential protein expression in isolated adipocytes from 3 month-old male WT, aP2-Cre, IR (lox/lox), and FIRKO mice. Adipocytes from epididymal fat pads of 4 WT and 8 FIRKO mice were isolated by collagenase digestion, pooled, and separated into two different subsets using a nylon mesh of 75 μm pore size. There was no difference in the expression of proteins between the two cell size subsets in adipocytes from the control mice (WT, IR (lox/lox), aP2-Cre) (data not shown). Therefore only the adipocyte cell size large (FIRKO L) and small (FIRKO S) FIRKO adipocytes are displayed (FIRKO L, adipocytes with a diameter >75 μm; FIRKO S, adipocytes with a diameter <75 μm). A representative Western blot and the data±SEM from four independent experiments are shown for (a) the insulin receptor, (b) GLUT1, (c) SREBP-1, (d) FAS, (e) C/EBPα, (f) IRS-1, (g) IRS-2, (h) GLUT4, (i) PPARγ, (j) leptin, (k) aP2. Insulin receptor and GLUT1 expression were decreased in both subsets of FIRKO adipocytes compared to all control groups. SREBP-1 and C/EBPα protein expression was decreased in FIRKO adipocytes compared to all control groups with significant higher levels in FIRKO L compared to the FIRKO S. The protein expression of FAS was not different between FIRKO L adipocytes and control groups, but significantly decreased in FIRKO S adipocytes. There were no significant differences in the IRS-1, IRS-2, GLUT-4, PPARγ, Leptin, and aP2 protein expression between the FIRKO L and FIRKO S subsets of adipocytes and between these two subsets and the adipocytes from the control groups.

FIG. 8 is a graph showing basal and insulin-stimulated glucose uptake in isolated adipocytes as a function of size in wild type vs. FIRKO mice.

FIG. 9 is a graph showing regulation of triglyceride synthesis as a function of size in wild type vs. FIRKO mice.

FIG. 10 is a graph showing basal lipolysis as a function of size in wild type vs. FIRKO mice.

FIG. 11 is a table showing differences in gene expression between large and small FIRKO adipocytes.

FIG. 12 is a table showing differences in protein expression between large and small FIRKO adipocytes.

FIG. 13 is a graph showing differential expression of mitochondrial genes in epididymal vs. subcutaneous fat.

### DETAILED DESCRIPTION

The data described herein show that adipocyte-specific reduction of IR signaling, e.g., disruption of the IR gene, produces selective insulin resistance in the adipose tissue, but does not affect whole body glucose metabolism. Lack of IR signaling in fat produces almost complete protection against age- and hyperphagia-associated obesity and the impairment of glucose tolerance associated with these conditions. While not wanting to be bound by theory, it is believed that selective reduction of IR signaling in fat tissue (a) inhibits lipogenesis or triglyceride storage in fat or increase lipolysis, thereby protecting against obesity and obesity related conditions and (b) unmasks an intrinsic heterogeneity in adipose cells, e.g., WAT cells, indicative of differences in subtypes of adipose cells related to differential function or development, e.g., within a single fat depot or among different fat depots.

Heterogeneity of Adipose Tissue

Inhibition of insulin signaling in fat tissue has unmasked a heterogeneity, e.g., an intrinsic heterogeneity, of the white adipose tissue. Distinct subpopulations of white adipose cells (e.g., the isolated populations of adipose cells described herein) have differential characteristics, e.g., one or more of: different size, different gene expression, and different function (e.g., different levels of glucose uptake, lipogenesis or lipolysis activity), e.g., in response to modulation of insulin signaling. In one embodiment, the characteristics of a subpopulation can be identified or unmasked by inhibition of insulin signaling, e.g., reduction in the expression, level or activity of a component of the insulin receptor signaling pathway, e.g., reduction of the levels or activity of insulin receptor (IR), insulin receptor substrate (IRS), phosphatidylinositol 3-kinase (PI3K), Akt, PKC, SHC, SHP-2, GRB2, SOS-1or Ras. The different subpopulations of white adipose cells can be indicative of different stages and/or branches of an adipose development pathway (e.g., differences in fat cell lineage development); of differences in function, e.g., different levels of glucose uptake, lipogenesis or lipolysis activity; or of differential gene expression or function of adipose cells from within a single depot or different depots.

The heterogeneity of adipocyte size in white adipose tissue in FIRKO mice suggests that specific adipocyte fractions are differentially affected by the IR knockout. The subset of small adipocytes (˜45% of the cells; e.g., TG-S cells) are protected from excessive TG load, whereas a second subset of FIRKO adipocytes maintain normal TG storage function. Thus, a knockout of the insulin receptor unmasks an intrinsic heterogeneity in adipocytes and that protection from excessive TG load in only a fraction of adipocytes is sufficient to protect FIRKO mice from development of obesity and its related effects on glucose intolerance and insulin resistance.

The development of the small and large subsets of FIRKO adipocytes was not due to inefficiency of the IR knockout. Likewise, there were no differences in the expression of the IRS proteins, the GLUT4 and GLUT1 glucose transporters, and the insulin-stimulated glucose uptake into adipocytes between these subsets of cells. Thus, differences in insulin signaling or glucose transport cannot explain the heterogeneity of the adipocyte size. One potential explanation for the heterogeneity in fat cell size of FIRKO mice might be that lipogenesis and differentiated phenotype are somehow differentially regulated in these adipocyte size fractions. This hypothesis is supported by the observation that small and large adipocytes from FIRKO mice differentially express fatty acid synthase and the adipogenic transcription factors SREBP-1 and C/EBPα, in each case with lower expression in the small adipocytes as compared to the large adipocytes. This heterogeneity might also represent different stages of adipocyte differentiation, although there were no differences in the protein levels of the adipogenesis markers PPARγ, GLUT4 and the adipocyte-fatty acid binding protein aP2, all features of terminal differentiated adipocytes. The differential protein expression patterns of SREBP-1, C/EBPα, and FAS in small and large FIRKO adipocytes might display a different susceptibility of these proteins to insulin regulation in different subsets of adipocytes or that differences in the timing of the IR knockout cause these differences in the protein expression.

FAS is normally upregulated by insulin through the phosphatidylinositol (PI)3-kinase signaling pathway and protein kinase B/akt as a downstream effector (Sul et al. (2000) J Nutr Feb;130(2S Suppl):315S-320S). The human FAS sequence is known and can be found in Jayakumar (1995) Proc. Natl. Acad. Sci. U.S.A. 92(19): 8695-8699.

Sterol regulatory element-binding proteins (SREBPs) are lipid synthetic transcription factors for cholesterol and fatty acid synthesis. SREBPs are synthesized as membrane-bound precursors with their N-terminal active portions entering the nucleus to activate target genes after proteolytic cleavage in a sterol-regulated manner. This cleavage step is regulated by a putative sterol-sensing molecule, SREBP-activating protein (SCAP), that forms a complex with SREBPs and traffics between the rough endoplasmic reticulum and Golgi. DNA cis-elements that SREBPs bind, originally identified as sterol-regulatory elements (SREs), now expands to a variety of SRE-like sequences and some of E-boxes, which makes SREBPs eligible to regulate a wide range of lipid genes. In differentiated tissues and organs, SREBP-1c is involved in fatty acid synthesis and seems to be a mediator for insulin/glucose signaling to lipogenesis, and could be involved in insulin resistance, remnant lipoproteins, and fatty livers (Shimano (2002) Vitam Horm 65:167-94).

C/EBPα has been implicated in adipocyte development (Lee et al. (1998) Mol Cell Biochem 178(1-2):269-74) and is overexpressed in adipose tissue of obese rats (Rolland et al. (1995) Biochem Biophys Res Commun 207(2):761-7).

FATP1 is involved in the uptake and transport of fatty acids across the cell membrane. FATP1 has been characterized in detail (Martin et al. (2000) Genomics 66(3):296-304). Insulin has previously been shown not to affect FATP-1 mRNA expression in skeletal muscle in obese nondiabetic or in type 2 diabetic subjects nor in subcutaneous adipose tissue, which has been interpreted to mean that FATP-1 expression may not contribute to a large extent to the alterations in fatty acid uptake in obesity and/or type 2 diabetes (Binnert et al. (2000) Am J Physiol Endocrinol Metab 279 (5):E1072-9).

Specific β3AR subtypes have been associated with obesity (Philipson (2002) J Allergy Clin Immunol 110(6 Suppl):S313-7). The presence of the P3AR in human white adipocytes is consistent with evidence that it can mediate lipolysis in human white adipocytes. The increased expression of the β3AR in obese subjects treated with caffeine and ephedrine has suggested the potential of β3AR agonists in the treatment of obesity and type 2 diabetes (De Matteis (2002) Int J Obes Relat Metab Disord 26(11): 1442-50).

Previous work by others has demonstrated that central (visceral) fat carries a high risk of diabetes and related conditions, whereas peripheral fat excess does not. Differences in these two depots have also been described based on rates of lipolysis and response to beta adrenergic agents. In addition to identifying specific subtypes of adipose cells within a single depot, the inventors have now found that different depots, e.g., visceral vs. peripheral depots, are composed of different kinds of fat cells which can be distinguished by specific differences in gene expression or markers and specifically targeted in disease. For example, expression of multiple mitochondrial genes, e.g., genes in the oxidative phosphorylation pathway, is reduced 2 in epididymal (visceral) as compared with subcutaneous adipocytes isolated from C57BL/6J mice, including 5 genes in complex IV of electron transport, UCP 1, and UCP 3.

Adipocyte Subtypes

The inventors have identified distinct subtypes of adipose cells that exhibit differential responses (e.g., differential gene expression, decreased size and/or changes in adipocyte cell function) to a modulation (preferably a reduction) in insulin signaling.

One such type of adipocyte (or subpopulation or subtype of adipocyte) described herein is a TG-S (Triglyceride-small) cell. TG-S cells, in the absence of insulin signaling (e.g., in the absence of a functional IR), can exhibit (a) a smaller than average size, e.g., less than 75 μm diameter, i.e., a low triglyceride load; (b) reduced expression of an enzyme, transcription factor or other protein involved in lipid synthesis or function, e.g., one or more of fatty acid synthase (FAS), SREBP-1c, C/EBPα, β3 adrenergic receptor, fatty acid transport protein 1 (FATP 1), fatty acid transport protein aP2 (FATP aP2), carnitine palmitoyltransferase 2, non-muscle myosin form A, acetyl CoA-dehydrogenase, citrate synthase, cytochrome C, vimentin, EH domain containing protein 2, elongation factor 2, glucose regulated protein 78 (GRP 78), transketolase, succinyl CoA transferase, activating transcription factor 3, TGFβ, PAI 1, annexin 2, annexin A6, 47 kDa heat shock protein, PDGF receptor, lumican and colony stimulating factor 3; and/or (c) decreased basal lipogenesis activity and/or isoproterenol induced lipolysis activity. TG-S cells can be, e.g., about 45% of the total adipocytes in a fat tissue in vivo.

A second cell or subpopulation or subtype of adipocyte described herein is a TG-L (triglyceride-large) cell. TG-L cells, in the absence of insulin signaling (e.g., in the absence of a functional IR), can exhibit (a) an average or larger than average size, e.g., greater than 100 μm diameter; and/or (b) increased basal lipogenesis and/or increased basal lipolysis activity.

Other types of subpopulation or subtype of adipocyte described herein include different types of adipose cells, e.g., WAT cells, present in different fat depots, which contribute to the different characteristics of the different depots. For example, visceral fat depots, which are associated with risk for diabetes or diabetes related conditions, are characterized by specific patterns of gene expression, e.g., in mitochondrial genes related to oxidative phosphorylation, e.g., a gene involved in oxidative phosphorylation described herein. For example, a visceral fat cell, e.g., WAT cell, can be characterized by reduced expression of one or more of: cytochrome p450 1b1, 17-beta-estradiol, angiotensinogen, thrombospondin 1, mevalonate kinase, prolactin receptor, or a protein in the mitochondrial oxidative phosphorylation pathway, e.g., cytochrome C oxidase subunit VIIIb, cytochrome C oxidase subunit VIIa, UCP1 or UCP3.

Insulin Signaling

Insulin is an essential regulator of intermediary metabolism and produces a broad spectrum of both direct and indirect effects in almost all tissues of the body. Tissue-specific disruption of insulin signaling has provided a powerful approach to dissect these complex and interacting pathways and to sort out direct and indirect effects of the hormone (Michael et al., 2000). It has been suggested that skeletal muscle accounts for 70-90% of glucose disposal following a carbohydrate load (DeFronzo, 1997), but the fraction of insulin stimulated glucose uptake in adipose tissue increases with duration of insulin elevation (James et al., 1985; Livingston et al., 1978). Fat clearly plays an important role in overall glucose homeostasis, however, as indicated by the insulin resistance associated with obesity (Kopelman, 2000) and various syndromes of lipodystrophy (Joffe et al., 2001), and the insulin resistance observed in mice with a fat-specific knockout of GLUT4 (Abel et al., 2001).

The phenotype of FIRKO mice is quite distinct from the phenotype of the adipocyte-selective reduction of glucose transporter GLUT4, which results in glucose intolerance, hyperinsulinemia and insulin resistance without an effect on adipose mass (Abel et al., 2001). While not wanting to be bound by theory, it is believed that the differences in the phenotype of FIRKO and adipose specific GLUT4 knockout mice may be explained by the fact that, in addition to the regulation of glucose transport, insulin has other important actions in adipose tissue, such as stimulation of lipogenesis, inhibition of lipolysis, and regulation of leptin secretion. These differences between the whole body glucose metabolism of the adipose tissue specific IR and GLUT4 knockout mice, as well as the differences observed between the muscle-specific IR (Brüning et al., 1998) and GLUT4 (Zisman et al., 2000) knockout mice further suggest that the level at which there is induction of insulin resistance even in a single tissue can contribute to major differences in phenotype. FIRKO mice, in which the IR is disrupted both in WAT and in BAT, also display a different phenotype from the brown adipose tissue-specific insulin receptor knockout (BATIRKO) (Guerra et al., 2001). The latter exhibit an age-dependent impaired glucose tolerance without insulin resistance, and this seems to be the primary result of a defect in insulin secretion. This indicates that the knockout of the insulin receptor in WAT has a protective effect over the glucose metabolism impairing effects of the IR knockout in BAT of BATIRKO mice, perhaps by altering one or more of the factors secreted by WAT.

Our data further show that insulin signaling in adipocytes is crucial for triglyceride storage and the development of obesity and its associated metabolic abnormalities. These insulin effects may be mediated by factors other than the impaired glucose transport in adipocytes, since fat-specific GLUT4 knockout mice have normal body weight, perigonadal fat pad weight and mean adipocyte size (Abel et al., 2001). The protection from obesity in FIRKO mice, despite the increased food intake relative to their body weight, could be explained by a permissive effect of insulin of triglyceride storage in fat or by the lack of antilipolytic insulin effects in adipocytes. Although plasma FFA, triglyceride, and lactate levels are not elevated in FIRKO mice, this does not preclude an increase in glycerol turnover due to increased lipolysis. Moreover, the resistance to obesity despite hyperphagia and the relative increase in UCP-1 expression in BAT of FIRKO mice suggest that metabolic rate is increased in FIRKO mice. By analogy to the BATIRKO mice, this may be the result of an increase in the thermogenic capacity of the BAT that contributes to the lean phenotype in FIRKO mice (Guerra et al., 2001).

Another surprising finding was the effect of the lack of insulin signaling in adipose tissue on morphology and protein expression in WAT. There was a marked reduction in GLUT1, but not in GLUT4, protein level in adipose tissue from FIRKO mice, indicating GLUT1 expression is directly insulin-regulated, whereas factors other than insulin are more important in the regulation of GLUT4 levels in vivo. This observation is in accordance with in vitro data showing that insulin selectively increases the amount of GLUT1 (Hajduch et al., 1992) in 3T3-L1 adipocytes without altering the GLUT4 expression and that dexamethasone-induced insulin resistance in these cells also acts primarily by causing a decrease in GLUT1 protein expression (Sakoda et al., 2000).

Leptin Regulation

FIRKO mice provide a novel model to investigate the role of insulin in the regulation of leptin secretion from adipose tissue in vivo. Since plasma leptin levels are normally proportional to adipose tissue mass (Maffei et al., 1995), we expected that FIRKO mice with a 50% decrease in adipose tissue mass would have proportional decreased plasma leptin levels. Despite the decreased body fat mass, however, plasma leptin levels are normal or slightly elevated in FIRKO mice, and markedly elevated when expressed as a function of body weight or fat mass. This finding is even more surprising since a lack of insulin signaling in adipocytes of FIRKO mice would be expected to lead to decreased plasma leptin levels, since both in vitro and clinical studies indicate that insulin stimulates leptin expression and secretion (D'Adamo et al., 1998; Bradley et al., 1999; Glasow et al., 2001). There is evidence for an interaction between leptin and insulin signaling pathways in vitro (Szanto et al., 2000; Zhao et al., 2000), and reduced glucose uptake in rat adipocytes has been shown to be associated with decreased leptin secretion in vitro (Mueller et al., 1998). However, our results in FIRKO mice confirm the previous finding in adipose selective GLUT4 knockout mice that normal glucose uptake into adipocytes is not necessary to maintain normal plasma leptin levels (Abel et al., 2001).

In summary, adipose selective reduction of IR signaling, e.g., knockout of the insulin receptor, protects against obesity and obesity-related glucose intolerance in animals, and leads to a loss of the normal relationship between leptin plasma concentration and body weight. Insulin receptor knockout in adipose tissue also causes a marked morphological change in white adipose tissue with heterogeneity of adipocyte size associated with changes in the protein expression pattern and ability of store triglycerides.

Modulation of the Insulin Receptor (IR) Signaling Pathway

An agent that reduces or increases signaling of the IR pathway described herein can affect the target specificity, stability, binding affinity to target, enzymatic activity (e.g., tyrosine kinase activity), susceptibility to regulation, and/or cofactor requirements of a component of the IR signaling pathway. For example, a variant of a component of the IR signaling pathway described herein (e.g., insulin, IR, IRS, PI3K, Akt, PKC, SHC, SHP-2, GRB2, SOS-1 or Ras) can have decreased or increased target specificity, stability, binding affinity to target, enzymatic activity, susceptibility to regulation, and/or cofactor requirements as compared to the native protein.

An inhibitor of the IR signaling pathway can be, e.g., an inhibitor of IR activity. Many examples of such inhibitors are known. For example, Grb14, a binding partner of IR, behaves as an uncompetitive inhibitor for the IR substrate and is a direct inhibitor of IR catalytic activity (Bereziat et al., 2002, J. Biol. Chem. 277:4845-52). The low molecular weight kinase inhibitor staurosporine is a selective inhibitor of IR tyrosine kinase activity (Fujita-Yamaguchi et al., 1988, Biochem Biophys Res Commun 157:955-62). Hydroxy-2-naphthalenyl-methyl phosphonic acid and its prodrug have been shown to inhibit insulin-stimulated autophosphorylation of IR, reducing IR function (Saperstein et al., 1989, Biochemistry 28:5694-701); Annexin I also inhibits IR autophosphorylation, specifically inhibiting insulin-stimulated IR tyrosine kinase activity (Melki et al., 1994, Biochem Biophys Res Commun 203:813-9). Human Alpha 2-HS glycoprotein (AHSG) inhibits the tyrosine kinase activity of IR in a dose-dependent fashion without interfering with the binding of insulin to IR. (Kalabay et al., 1998, Horm Metab Res 30:1-6). Catecholamines and tumour promoting phorbolesters also inhibit the kinase activity of IR (Obermaier et al., 1987, Diabetologia 30:93-9). In another example, activation of PKC isoforms β1 and β2 has also been shown to inhibit IR signaling (Bossenmaier et al., 1997, Diabetologia 40:863-6).

Other inhibitors of IR include inactivating anti-IR antibodies. For example, production of antibodies that inhibit the binding of insulin to IR are described in Roth et al. (1981) Biochem Biophys Res Commun 101:979-87; and Roth et al. (1982) PNAS U.S.A. 79:7312-6.

Inhibitors of the IR or other components of the insulin receptor signaling pathway, e.g., inhibitors described herein, include naturally occurring or synthetic polypeptides; naturally occurring or synthetic nucleic acids; naturally occurring or synthetic chemical compounds, e.g., organic compounds. Thus, one of skill in the art could look to libraries or other sources of each of these kinds of molecules (e.g., natural substance banks, combinatorial chemistry, phage display libraries) to screen for putative inhibitors of the insulin receptor signaling pathway. Methods for generating fragments, variants, chemical compounds, and testing them for the desired activity (e.g., the methods described herein below) are known in the art.

Targeting of Agents to Adipose Tissue

A number of strategies are available to one skilled in the art to target agents that reduce or increase insulin receptor signaling to adipose tissue, e.g., WAT. For example, nucleic acids that can inhibit expression of a component of the IR signaling pathway (e.g., IR, IRS, Grb2, SOS-1, Ras) can be placed under the control of an adipocyte specific control region, e.g., a promoter and/or enhancer, such that the nucleic acid is expressed selectively in adipose tissue. Alternatively, if it is desired to increase IR signaling in an adipocyte, a nucleic acid that can increase expression of (e.g., encodes) a component of the IR signaling pathway (e.g., insulin, IR, IRS, PI3K, Akt, PKC, SHC, SHP-2, GRB2, SOS-1 or Ras, or a functional fragment thereof) can be placed under the control of an adipocyte specific control region, e.g., a promoter and/or enhancer, such that the nucleic acid is expressed selectively in adipose tissue. Adipocyte-specific control regions are known in the art. Examples are described herein below.

In other embodiments, an agent that reduces or increases insulin receptor signaling can be targeted to adipose tissue by using prodrug strategies, e.g., antibody-directed, gene-directed or virus-directed enzyme prodrug therapy. In other embodiments, an agent is targeted to adipose tissue by combining the agent (e.g., linking, fusing, conjugating or enveloping the agent) with a targeting reagent that is targeted, preferably specifically, to an adipose tissue.

Adipose Tissue-Specific Control Regions

Adipose tissue-specific promoters which provide expression in an adipocyte, e.g., a WAT adipocyte, can be used in the methods described herein. Adipocyte specific promoters are promoters which are expressed more strongly in adipocytes than in other tissues, e.g., adipocyte specific promoters can be expressed essentially exclusively in the adipose tissue. Many adipocyte-specific promoters which can be used in the methods described herein are known.

For example, the human adipocyte-specific apM-1 gene encodes a secretory protein of the adipose tissue. Several binding sites known to be involved in adipogenesis and regulation of adipocyte-specific genes are present in the proximal promoter region of apM-1, which has been cloned and characterized (see, e.g., Schaffler et al. (1998) Biochim Biophys Acta 1399:187-97).

As leptin is expressed only in mature adipose cells, its promoter can also be used in tissue-specific targeting of nucleic acids. The leptin gene (ob) promoter has been cloned and it has been found that the adipocyte-specific transcription factor CCAAT-enhancer-binding-protein-alpha (C/EBPalpha) modulates human ob gene expression (see Miller et al., 1996, PNAS USA 93:5507-11). Accordingly, the placement of an C/EBPalpha binding site upstream of a nucleic acid desired to be expressed selectively in adipose tissue can be used in the methods described herein.

Another adipocyte specific enhancer activates the phosphoenolpyruvate carboxykinase (PEPCK) gene in adipocytes. The nuclear receptor, PPAR-gamma (as a heterodimer with retinoid X receptor, RXR), activates this enhancer. The adipocyte-specific enhancer has been mapped to approximately 1 kb upstream of the PEPCK gene. A 413-base pair region between −1242 and -828 bp can be used as an adipocyte-specific enhancer in vivo (see, e.g., Devine et al. (1999) J Biol Chem 274:13604-12).

In addition, the promoters of genes encoding enzymes involved in fatty acid synthesis, e.g., stearoyl-CoA desaturase 1 (SCD1) (Ntambi et al., 1988, J. Biol. Chem. 263, 17291-17300); SCD2 (Kaestner, 1989, J Biol. Chem. 264:14755-61), and fatty acid synthase (FAS), can also be used in the methods described herein. Other adipocyte-specific control regions include those of adipose P2 (aP2) and adipsin (both described in U.S. Pat. No. 5,476,926); p154 (described in U.S. Pat. No. 5,541,068); and adipocyte-specific differentiation-related protein (HADRP) (described in U.S. Pat. No. 5,739,009).

Adipocyte-Specific Targeting Reagents

An agent that increases or decreases IR signaling, e.g., an agent described herein, can be targeted to adipose tissue by combining the agent (e.g., linking, fusing, conjugating or enveloping the agent) with a targeting reagent that is targeted, preferably specifically, to an adipose tissue. Examples of such reagents are known and include, e.g., leptin conjugates, liposomes, antibodies directed to adipocyte-specific surface antigens. The agent and targeting reagent are preferably lipid soluble.

Other methods for targeting agents to cells of choice, which could be generally applied to adipocytes, are described, e.g., in Economides (1995) Science 270:1351-3.

Antisense Nucleic Acid Sequences

Nucleic acid molecules which are antisense to a nucleotide encoding a component of the IR signaling pathway described herein, e.g., a component described herein, can also be used as an agent which inhibits expression of the component of the IR signaling pathway. An “antisense” nucleic acid includes a nucleotide sequence which is complementary to a “sense” nucleic acid encoding the component, e.g., complementary to the coding strand of a double-stranded cDNA molecule or complementary to an mRNA sequence. Accordingly, an antisense nucleic acid can form hydrogen bonds with a sense nucleic acid. The antisense nucleic acid can be complementary to an entire coding strand, or to only a portion thereof. For example, an antisense nucleic acid molecule which antisense to the “coding region” of the coding strand of a nucleotide sequence encoding the component can be used.

The coding strand sequences encoding the components of the IR signaling pathway described herein are known. Given the coding strand sequences encoding these proteins, antisense nucleic acids can be designed according to the rules of Watson and Crick base pairing. The antisense nucleic acid molecule can be complementary to the entire coding region of mRNA, but more preferably is an oligonucleotide which is antisense to only a portion of the coding or noncoding region of mRNA. For example, the antisense oligonucleotide can be complementary to the region surrounding the translation start site of the mRNA. An antisense oligonucleotide can be, for example, about 5, 10, 15, 20, 25, 30, 35, 40, 45 or 50 nucleotides in length. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest.

RNAi

Double stranded nucleic acid molecules that can silence a gene encoding a component of the IR signaling pathway described herein, e.g., a component described herein, can also be used as an agent which inhibits expression of the component of the IR signaling pathway. RNA interference (RNAi) is a mechanism of post-transcriptional gene silencing in which double-stranded RNA (dsRNA) corresponding to a gene (or coding region) of interest is introduced into a cell or an organism, resulting in degradation of the corresponding mRNA. The RNAi effect persists for multiple cell divisions before gene expression is regained. RNAi is therefore an extremely powerful method for making targeted knockouts or “knockdowns” at the RNA level. RNAi has proven successful in human cells, including human embryonic kidney and HeLa cells (see, e.g., Elbashir et al. Nature 2001 May 24;411(6836):494-8). In one embodiment, gene silencing can be induced in mammalian cells by enforcing endogenous expression of RNA hairpins (see Paddison et al.,2002, PNAS USA 99:1443-1448). In another embodiment, transfection of small (21-23 nt) dsRNA specifically inhibits gene expression (reviewed in Caplen (2002) Trends in Biotechnology 20:49-51).

Briefly, RNAi is thought to work as follows. dsRNA corresponding to a portion of a gene to be silenced is introduced into a cell. The dsRNA is digested into 21-23 nucleotide siRNAs, or short interfering RNAs. The siRNA duplexes bind to a nuclease complex to form what is known as the RNA-induced silencing complex, or RISC. The RISC targets the homologous transcript by base pairing interactions between one of the siRNA strands and the endogenous mRNA. It then cleaves the mRNA ˜12 nucleotides from the 3′ terminus of the siRNA (reviewed in Sharp et al (2001) Genes Dev 15: 485-490; and Hammond et al. (2001) Nature Rev Gen 2: 110-119).

RNAi technology in gene silencing utilizes standard molecular biology methods. dsRNA corresponding to the sequence from a target gene to be inactivated can be produced by standard methods, e.g., by simultaneous transcription of both strands of a template DNA (corresponding to the target sequence) with T7 RNA polymerase. Kits for production of dsRNA for use in RNAi are available commercially, e.g., from New England Biolabs, Inc. Methods of transfection of dsRNA or plasmids engineered to make dsRNA are routine in the art.

Gene silencing effects similar to those of RNAi have been reported in mammalian cells with transfection of a mRNA-cDNA hybrid construct (Lin et al., Biochem Biophys Res Commun 2001 Mar. 2;281(3):639-44), providing yet another strategy for gene silencing.

Peptide Mimetics

The invention also provides for production of the protein binding domains of components of the IR signaling pathway, e.g., insulin, IR, IRS, PI3K, AKT, PKC, SHC, SHP-2, GRB2, SOS-1 or Ras, to generate mimetics, e.g. peptide or non-peptide agents, e.g., inhibitory agents. See, for example, “Peptide inhibitors of human papillomavirus protein binding to retinoblastoma gene protein” European patent applications EP 0 412 762 and EP 0 031 080.

Non-hydrolyzable peptide analogs of critical residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), b-turn dipeptide cores (Nagai et al. (1985) Tetrahedron Lett 26:647; and Sato et al. (1986) J Chem Soc Perkin Trans 1:1231), and b-aminoalcohols (Gordon et al. (1985) Biochem Biophys Res Commun126:419; and Dann et al. (1986) Biochem Biophys Res Commun 134:71).

Antibodies

An agent described herein, e.g., an agent that inhibits or promotes signaling through the IR signaling pathway, can also be an antibody specifically reactive with an alternative pathway component, e.g., insulin, IR, IRS, PI3K, AKT, PKC, SHC, SHP-2, GRB2, SOS-1 or Ras. An antibody can be an antibody or a fragment thereof, e.g., an antigen binding portion thereof. As used herein, the term “antibody” refers to a protein comprising at least one, and preferably two, heavy (H) chain variable regions (abbreviated herein as VH), and at least one and preferably two light (L) chain variable regions (abbreviated herein as VL). The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4.

The antibody can further include a heavy and light chain constant region, to thereby form a heavy and light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (C1q) of the classical complement system.

The term “antigen-binding fragment” of an antibody (or simply “antibody portion,” or “fragment”), as used herein, refers to one or more fragments of a full-length antibody that retain the ability to specifically bind to an antigen (e.g., a polypeptide encoded by a nucleic acid of Group I or II). Examples of binding fragments encompassed within the term “antigen-binding fragment” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody,
(v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate nucleic acids, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies. The term “monoclonal antibody” or “monoclonal antibody composition”, as used herein, refers to a population of antibody molecules that contain only one species of an antigen binding site capable of immunoreacting with a particular epitope. A monoclonal antibody composition thus typically displays a single binding affinity for a particular protein with which it immunoreacts.

Anti-protein/anti-peptide antisera or monoclonal antibodies can be made as described herein by using standard protocols (See, for example, Antibodies: A Laboratory Manual ed. by Harlow and Lane (Cold Spring Harbor Press: 1988)).

A components of the IR signaling pathway, e.g., insulin, IR, IRS, PI3K, AKT, PKC, SHC, SHP-2, GRB2, SOS-1 or Ras, or a portion or fragment thereof, can be used as an immunogen to generate antibodies that bind the component using standard techniques for polyclonal and monoclonal antibody preparation. The full-length component protein can be used or, alternatively, antigenic peptide fragments of the component can be used as immunogens.

Typically, a peptide is used to prepare antibodies by immunizing a suitable subject, (e.g., rabbit, goat, mouse or other mammal) with the immunogen. An appropriate immunogenic preparation can contain, for example, a recombinant component of the IR signaling pathway, e.g., insulin, IR, IRS, PI3K, AKT, PKC, SHC, SHP-2, GRB2, SOS-1 or Ras peptide, or a chemically synthesized component of the IR signaling pathway, e.g., insulin, IR, IRS, P13K, SHC, SHP-2, GRB2, SOS-1 or Ras peptide or anagonist. See, e.g., U.S. Pat. No. 5,460,959; and co-pending U.S. applications U.S. Ser. No. 08/334,797; U.S. Ser. No. 08/231,439; U.S. Ser. No. 08/334,455; and U.S. Ser. No. 08/928,881, which are hereby expressly incorporated by, reference in their entirety. The nucleotide and amino acid sequences of the alternative pathway components, e.g., insulin, IR, IRS, P13K, AKT, PKC, SHC, SHP-2, GRB2, SOS-1 or Ras, are known. The preparation can further include an adjuvant, such as Freund's complete or incomplete adjuvant, or similar immunostimulatory agent. Immunization of a suitable subject with an immunogenic component of the IR signaling pathway, e.g., insulin, IR, IRS, PI3K, AKT, PKC, SHC, SHP-2, GRB2, SOS-1 or Ras, or fragment preparation induces a polyclonal antibody response.

Additionally, antibodies produced by genetic engineering methods, such as chimeric and humanized monoclonal antibodies, comprising both human and non-human portions, which can be made using standard recombinant DNA techniques, can be used. Such chimeric and humanized monoclonal antibodies can be produced by genetic engineering using standard DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al., Science 240:1041-1043, 1988; Liu et al., PNAS 84:3439-3443, 1987; Liu et al., J. Immunol. 139:3521-3526, 1987; Sun et al. PNAS 84:214-218, 1987; Nishimura et al., Canc. Res. 47:999-1005, 1987; Wood et al., Nature 314:446-449, 1985; and Shaw et al., J. Natl. Cancer Inst. 80:1553-1559, 1988); Morrison, S. L., Science 229:1202-1207, 1985; Oi et al., BioTechniques 4:214, 1986; Winter U.S. Pat. No. 5,225,539; Jones et al., Nature 321:552-525, 1986; Verhoeyan et al., Science 239:1534, 1988; and Beidler et al., J. Immunol. 141:4053-4060, 1988.

In addition, a human monoclonal antibody directed against a component of the IR signaling pathway, e.g., insulin, IR, IRS, PI3K, AKT, PKC, SHC, SHP-2, GRB2, SOS-1 or Ras, can be made using standard techniques. For example, human monoclonal antibodies can be generated in transgenic mice or in immune deficient mice engrafted with antibody-producing human cells. Methods of generating such mice are describe, for example, in Wood et al. PCT publication WO 91/00906, Kucherlapati et al. PCT publication WO 91/10741; Lonberg et al. PCT publication WO 92/03918; Kay et al. PCT publication WO 92/03917; Kay et al. PCT publication WO 93/12227; Kay et al. PCT publication 94/25585; Rajewsky et al. Pct publication WO 94/04667; Ditullio et al. PCT publication WO 95/17085; Lonberg, N. et al. (1994) Nature 368:856-859; Green, L. L. et al. (1994) Nature Genet. 7:13-21; Morrison, S. L. et al. (1994) Proc. Natl. Acad. Sci. USA 81:6851-6855; Bruggeman et al. (1993) Year Immunol 7:33-40; Choi et al. (1993) Nature Genet. 4:117-123; Tuaillon et al. (1993) PNAS 90:3720-3724; Bruggeman et al. (1991) Eur J Immunol 21:1323-1326); Duchosal et al. PCT publication WO 93/05796; U.S. Pat. No. 5,411,749; McCune et al. (1988) Science 241:1632-1639), Kamel-Reid et al. (1988) Science 242:1706; Spanopoulou (1994) Genes & Development 8:1030-1042; Shinkai et al. (1992) Cell 68:855-868). A human antibody-transgenic mouse or an immune deficient mouse engrafted with human antibody-producing cells or tissue can be immunized with a component of the IR signaling pathway, e.g., insulin, IR, IRS, PI3K, AKT, PKC, SHC, SHP-2, GRB2, SOS-1 or Ras, or an antigenic peptide thereof, and splenocytes from these immunized mice can then be used to create hybridomas. Methods of hybridoma production are well known.

Human monoclonal antibodies can also be prepared by constructing a combinatorial immunoglobulin library, such as a Fab phage display library or a scFv phage display library, using immunoglobulin light chain and heavy chain cDNAs prepared from mRNA derived from lymphocytes of a subject. See, e.g., McCafferty et al. PCT publication WO 92/01047; Marks et al. (1991) J. Mol. Biol. 222:581-597; and Griffths et al. (1993) EMBO J. 12:725-734. In addition, a combinatorial library of antibody variable regions can be generated by mutating a known human antibody. For example, a variable region of a human antibody known to bind a component of the IR signaling pathway, e.g., insulin, IR, IRS, PI3K, AKT, PKC, SHC, SHP-2, GRB2, SOS-1 or Ras, can be mutated, by for example using randomly altered mutagenized oligonucleotides, to generate a library of mutated variable regions which can then be screened to bind to a component of the IR signaling pathway, e.g., a component described herein. Methods of inducing random mutagenesis within the CDR regions of immunoglobin heavy and/or light chains, methods of crossing randomized heavy and light chains to form pairings and screening methods can be found in, for example, Barbas et al. PCT publication WO 96/07754; Barbas et al. (1992) Proc. Nat'l Acad. Sci. USA 89:4457-4461.

The immunoglobulin library can be expressed by a population of display packages, preferably derived from filamentous phage, to form an antibody display library. Examples of methods and reagents particularly amenable for use in generating antibody display library can be found in, for example, Ladner et al. U.S. Pat. No. 5,223,409; Kang et al. PCT publication WO 92/18619; Dower et al. PCT publication WO 91/17271; Winter et al. PCT publication WO 92/20791; Markland et al. PCT publication WO 92/15679; Breitling et al. PCT publication WO 93/01288; McCafferty et al. PCT publication WO 92/01047; Garrard et al. PCT publication WO 92/09690; Ladner et al. PCT publication WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) supra; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982. Once displayed on the surface of a display package (e.g., filamentous phage), the antibody library is screened to identify and isolate packages that express an antibody that binds a component of the IR signaling pathway. In a preferred embodiment, the primary screening of the library involves panning with an immobilized alternative pathway component described herein and display packages expressing antibodies that bind immobilized proteins described herein are selected.

Transgenic Animals

The invention provides non-human transgenic animals. As used herein, a “transgenic animal” is a non-human animal, preferably a mammal, e.g., a rodent such as a rat or mouse, a meat mammal such as a hog, goat or beef cattle, in which one or more of the cells of the animal includes a transgene. Other examples of transgenic animals include non-human primates, sheep, dogs, chickens, amphibians, and the like. A transgene is exogenous DNA or a rearrangement, e.g., a deletion of endogenous chromosomal DNA, which preferably is integrated into or occurs in the genome of the cells of a transgenic animal. A transgene can direct the expression of an encoded gene product in one or more cell types or tissues of the transgenic animal, other transgenes, e.g., a knockout, reduce expression. Thus, a transgenic animal can be one in which an endogenous IR gene (or other component of the IR signaling pathway described herein) has been altered by, e.g., by homologous recombination between the endogenous gene and an exogenous DNA molecule introduced into a cell of the animal, e.g., an embryonic cell of the animal, prior to development of the animal. In preferred embodiments, the gene is altered in a tissue specific, e.g., adipose tissue, e.g., WAT-specific manner.

Intronic sequences and polyadenylation signals can also be included in the transgene to increase the efficiency of expression of the transgene. A tissue-specific (e.g., adipose specific, e.g.,. WAT-specific) regulatory sequence(s) can be operably linked to a transgene of the invention to direct expression of a protein to particular cells, e.g., adipose cells. A transgenic founder animal can be identified based upon the presence of a transgene in its genome and/or expression of the expressed mRNA in tissues or cells (e.g., adipose tissue) of the animals. A transgenic founder animal can then be used to breed additional animals carrying the transgene. Moreover, transgenic animals carrying a transgene encoding a desired protein can further be bred to other transgenic animals carrying other transgenes. In preferred embodiments a nucleic acid is placed under the control of a tissue specific promoter, e.g., an adipose tissue-specific promoter, Suitable animals are mice, pigs, cows, goats, dogs, cats, rats.

In some embodiment, a transgenic animal can be engineered such that a site specific recombination enzyme activates a transgenic sequence specifically in an adipose tissue. For example, a transgenic animal is created in which site-specific DNA recombination sites, e.g., loxP sites, are inserted so they flank the gene of interest or an essential exon. A transgenic animal is also prepared which carries a nucleotide sequence encoding an enzyme that catalyzes recombination, e.g., Cre, linked to a cell-type-specific promoter, e.g., an adipose-specific promoter described herein. Mating of these two types of animal will yield progeny that carry the sequence of interest modified by insertion of flanking lox P sites and the cre gene controlled by a cell-type-specific promoter. In these animals, recombination between the loxP sites, which disrupts the gene of interest, will occur only in those cells in which the promoter is active and therefore producing the Cre protein necessary to induce the recombination, producing a transgenic animal having an adipose-specific disruption of a particular gene, e.g., a gene of a component of the IR signaling pathway, e.g., insulin, IR, IRS, Sch, SH-2, SOS-1, Grb2.

The invention also includes a population of cells from a transgenic animal.

Techniques for production of transgenic animals are known in the art. For example, specific guidance on the production of transgenic animals is provided in: Gene Knockout Protocols (Tymms and Kola, Eds., Humana Press, 2001); Gene Targeting, A Practical Approach (Joyner, Ed., Oxford University press, 2000); Transgenic Animal Technology: A Laboratory Handbook (Pinkert, Ed., Academic Press, 1984).

Generation of Variants: Production of Altered DNA and Peptide Sequences by Random Methods

Methods are provided herein below for the production of variants of components of the IR signaling pathway, e.g., insulin, IR, IRS, PI3K, SHC, SHP-2, GRB2, SOS-1, or Ras, and for the screening of such variants for a desired activity. Amino acid sequence variants of a component of the IR signaling pathway, e.g., insulin, IR, IRS, P13K, SHC, SHP-2, GRB2, SOS-1, Ras, or fragments thereof, can be prepared by random mutagenesis of DNA which encodes a component of the IR signaling pathway, e.g., insulin, IR, IRS, PI3K, SHC, SHP-2, GRB2, SOS-1 or Ras. Useful methods include PCR mutagenesis and saturation mutagenesis. A library of random amino acid sequence variants can also be generated by the synthesis of a set of degenerate oligonucleotide sequences. One of ordinary skill in the art can use these methods to produce and screen a library, e.g., a library described herein, for the ability to inhibit or promote IR signaling. Assays that can be used to determine if a particular variant has the ability to inhibit or promote IR signaling are also provided herein below.

PCR Mutagenesis

In PCR mutagenesis, reduced Taq polymerase fidelity is used to introduce random mutations into a cloned fragment of DNA (Leung et al., 1989, Technique 1:11-15). This is a very powerful and relatively rapid method of introducing random mutations. The DNA region to be mutagenized is amplified using the polymerase chain reaction (PCR) under conditions that reduce the fidelity of DNA synthesis by Taq DNA polymerase, e.g., by using a dGTP/dATP ratio of five and adding Mn⁺² to the PCR reaction. The pool of amplified DNA fragments are inserted into appropriate cloning vectors to provide random mutant libraries.

Saturation Mutagenesis

Saturation mutagenesis allows for the rapid introduction of a large number of single base substitutions into cloned DNA fragments (Mayers et al., 1985, Science 229:242). This technique includes generation of mutations, e.g., by chemical treatment or irradiation of single-stranded DNA in vitro, and synthesis of a complimentary DNA strand. The mutation frequency can be modulated by modulating the severity of the treatment, and essentially all possible base substitutions can be obtained. Because this procedure does not involve a genetic selection for mutant fragments both neutral substitutions, as well as those that alter function, are obtained. The distribution of point mutations is not biased toward conserved sequence elements.

Degenerate Oligonucleotides

A library of homologs can also be generated from a set of degenerate oligonucleotide sequences. Chemical synthesis of a degenerate sequences can be carried out in an automatic DNA synthesizer, and the synthetic genes then ligated into an appropriate expression vector. The synthesis of degenerate oligonucleotides is known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Generation of Variants: Production of Altered DNA and Peptide Sequences by Directed Mutagenesis

Non-random or directed mutagenesis techniques can be used to provide specific sequences or mutations in specific regions. These techniques can be used to create variants that include, e.g., deletions, insertions, or substitutions, of residues of the known amino acid sequence of a protein. The sites for mutation can be modified individually or in series, e.g., by (1) substituting first with conserved amino acids and then with more radical choices depending upon results achieved, (2) deleting the target residue, or (3) inserting residues of the same or a different class adjacent to the located site, or combinations of options 1-3.

Alanine Scanning Mutagenesis

Alanine scanning mutagenesis is a useful method for identification of certain residues or regions of the desired protein that are preferred locations or domains for mutagenesis, Cunningham and Wells (Science 244:1081-1085, 1989). In alanine scanning, a residue or group of target residues are identified (e.g., charged residues such as Arg, Asp, His, Lys, and Glu) and replaced by a neutral or negatively charged amino acid (most preferably alanine or polyalanine). Replacement of an amino acid can affect the interaction of the amino acids with the surrounding aqueous environment in or outside the cell. Those domains demonstrating functional sensitivity to the substitutions are then refined by introducing further or other variants at or for the sites of substitution. Thus, while the site for introducing an amino acid sequence variation is predetermined, the nature of the mutation per se need not be predetermined. For example, to optimize the performance of a mutation at a given site, alanine scanning or random mutagenesis may be conducted at the target codon or region and the expressed desired protein subunit variants are screened for the optimal combination of desired activity.

Oligonucleotide-Mediated Mutagenesis

Oligonucleotide-mediated mutagenesis is a useful method for preparing substitution, deletion, and insertion variants of DNA, see, e.g., Adelman et al., (DNA 2:183, 1983). Briefly, the desired DNA is altered by hybridizing an oligonucleotide encoding a mutation to a DNA template, where the template is the single-stranded form of a plasmid or bacteriophage containing the unaltered or native DNA sequence of the desired protein. After hybridization, a DNA polymerase is used to synthesize an entire second complementary strand of the template that will thus incorporate the oligonucleotide primer, and will code for the selected alteration in the desired protein DNA. Generally, oligonucleotides of at least 25 nucleotides in length are used. An optimal oligonucleotide will have 12 to 15 nucleotides that are completely complementary to the template on either side of the nucleotide(s) coding for the mutation. This ensures that the oligonucleotide will hybridize properly to the single-stranded DNA template molecule. The oligonucleotides are readily synthesized using techniques known in the art such as that described by Crea et al. (Proc. Natl. Acad. Sci. (1978) USA, 75: 5765).

Cassette Mutagenesis

Another method for preparing variants, cassette mutagenesis, is based on the technique described by Wells et al. (Gene, 34:315[1985]). The starting material is a plasmid (or other vector) which includes the protein subunit DNA to be mutated. The codon(s) in the protein subunit DNA to be mutated are identified. There must be a unique restriction endonuclease site on each side of the identified mutation site(s). If no such restriction sites exist, they may be generated using the above-described oligonucleotide-mediated mutagenesis method to introduce them at appropriate locations in the desired protein subunit DNA. After the restriction sites have been introduced into the plasmid, the plasmid is cut at these sites to linearize it. A double-stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are comparable with the ends of the linearized plasmid, such that it can be directly ligated to the plasmid. This plasmid now contains the mutated desired protein subunit DNA sequence.

Combinatorial Mutagenesis

Combinatorial mutagenesis can also be used to generate mutants. For example, the amino acid sequences for a group of homologs or other related proteins are aligned, preferably to promote the highest homology possible. All of the amino acids which appear at a given position of the aligned sequences can be selected to create a degenerate set of combinatorial sequences. The variegated library of variants is generated by combinatorial mutagenesis at the nucleic acid level, and is encoded by a variegated gene library. For example, a mixture of synthetic oligonucleotides can be enzymatically ligated into gene sequences such that the degenerate set of potential sequences are expressible as individual peptides, or alternatively, as a set of larger fusion proteins containing the set of degenerate sequences.

Primary High-Through-Put Methods for Screening Libraries of Peptide Fragments or Homologs

Various techniques are known in the art for screening peptides, e.g., synthetic peptides, e.g., small molecular weight peptides (e.g., linear or cyclic peptides) or generated mutant gene products. Techniques for screening large gene libraries often include cloning the gene library into replicable expression vectors, transforming appropriate cells with the resulting library of vectors, and expressing the genes under conditions in which detection of a desired activity, assembly into a trimeric molecules, binding to natural ligands, e.g., a receptor or substrates, facilitates relatively easy isolation of the vector encoding the gene whose product was detected. Each of the techniques described below is amenable to high through-put analysis for screening large numbers of sequences created, e.g., by random mutagenesis techniques.

Two Hybrid Systems

Two hybrid (interaction trap) assays can be used to identify a protein that interacts with a component of the IR signaling pathway, e.g., insulin, IR, IRS, PI3K, SHC, SHP-2, GRB2, SOS-1, Ras or active fragments thereof. These may include, e.g., agonists, superagonists, and antagonists of insulin, IR, IRS, PI3K, SHC, SHP-2, GRB2, SOS-1, Ras. (The subject protein and a protein it interacts with are used as the bait protein and fish proteins.). These assays rely on detecting the reconstitution of a functional transcriptional activator mediated by protein-protein interactions with a bait protein. In particular, these assays make use of chimeric genes which express hybrid proteins. The first hybrid comprises a DNA-binding domain fused to the bait protein, e.g., insulin, IR, IRS, PI3K, SHC, SHP-2, GRB2, SOS-1, Ras or active fragments thereof. The second hybrid protein contains a transcriptional activation domain fused to a “fish” protein, e.g. an expression library. If the fish and bait proteins are able to interact, they bring into close proximity the DNA-binding and transcriptional activator domains. This proximity is sufficient to cause transcription of a reporter gene which is operably linked to a transcriptional regulatory site which is recognized by the DNA binding domain, and expression of the marker gene can be detected and used to score for the interaction of the bait protein with another protein.

Display Libraries

In one approach to screening assays, the candidate peptides are displayed on the surface of a cell or viral particle, and the ability of particular cells or viral particles to bind an appropriate receptor protein via the displayed product is detected in a “panning assay”. For example, the gene library can be cloned into the gene for a surface membrane protein of a bacterial cell, and the resulting fusion protein detected by panning (Ladner et al., WO 88/06630; Fuchs et al. (1991) Bio/Technology 9:1370-1371; and Goward et al. (1992) TIBS 18:136-140). This technique was used in Sahu et al. (1996) J. Immunology 157:884-891, to isolate a complement inhibitor. In a similar fashion, a detectably labeled ligand can be used to score for potentially functional peptide homologs. Fluorescently labeled ligands, e.g., receptors, can be used to detect homolog which retain ligand-binding activity. The use of fluorescently labeled ligands, allows cells to be visually inspected and separated under a fluorescence microscope, or, where the morphology of the cell permits, to be separated by a fluorescence-activated cell sorter.

A gene library can be expressed as a fusion protein on the surface of a viral particle. For instance, in the filamentous phage system, foreign peptide sequences can be expressed on the surface of infectious phage, thereby conferring two significant benefits. First, since these phage can be applied to affinity matrices at concentrations well over 10¹³ phage per milliliter, a large number of phage can be screened at one time. Second, since each infectious phage displays a gene product on its surface, if a particular phage is recovered from an affinity matrix in low yield, the phage can be amplified by another round of infection. The group of almost identical E. coli filamentous phages M13, fd., and f1 are most often used in phage display libraries. Either of the phage gIII or gVIII coat proteins can be used to generate fusion proteins without disrupting the ultimate packaging of the viral particle. Foreign epitopes can be expressed at the NH₂-terminal end of pIII and phage bearing such epitopes recovered from a large excess of phage lacking this epitope (Ladner et al. PCT publication WO 90/02909; Garrard et al., PCT publication WO 92/09690; Marks et al. (1992) J. Biol. Chem. 267:16007-16010; Griffiths et al. (1993) EMBO J. 12:725-734; Clackson et al. (1991) Nature 352:624-628; and Barbas et al. (1992) PNAS 89:4457-4461).

A common approach uses the maltose receptor of E. coli (the outer membrane protein, LamB) as a peptide fusion partner (Charbit et al. (1986) EMBO 5, 3029-3037). Oligonucleotides have been inserted into plasmids encoding the LamB gene to produce peptides fused into one of the extracellular loops of the protein. These peptides are available for binding to ligands, e.g., to antibodies, and can elicit an immune response when the cells are administered to animals. Other cell surface proteins, e.g., OmpA (Schorr et al. (1991) Vaccines 91, pp. 387-392), PhoE (Agterberg, et al. (1990) Gene 88, 37-45), and PAL (Fuchs et al. (1991) Bio/Tech 9, 1369-1372), as well as large bacterial surface structures have served as vehicles for peptide display. Peptides can be fused to pilin, a protein which polymerizes to form the pilus-a conduit for interbacterial exchange of genetic information (Thiry et al. (1989) Appl. Environ. Microbiol. 55, 984-993). Because of its role in interacting with other cells, the pilus provides a useful support for the presentation of peptides to the extracellular environment. Another large surface structure used for peptide display is the bacterial motive organ, the flagellum. Fusion of peptides to the subunit protein flagellin offers a dense array of may peptides copies on the host cells (Kuwajima et al. (1988) Bio/Tech. 6, 1080-1083). Surface proteins of other bacterial species have also served as peptide fusion partners. Examples include the Staphylococcus protein A and the outer membrane protease IgA of Neisseria (Hansson et al. (1992) J. Bacteriol. 174, 4239-4245 and Klauser et al. (1990) EMBO J. 9, 1991-1999).

In the filamentous phage systems and the LamB system described above, the physical link between the peptide and its encoding DNA occurs by the containment of the DNA within a particle (cell or phage) that carries the peptide on its surface. Capturing the peptide captures the particle and the DNA within. An alternative scheme uses the DNA-binding protein LacI to form a link between peptide and DNA (Cull et al. (1992) PNAS USA 89:1865-1869). This system uses a plasmid containing the LacI gene with an oligonucleotide cloning site at its 3′-end. Under the controlled induction by arabinose, a LacI-peptide fusion protein is produced. This fusion retains the natural ability of LacI to bind to a short DNA sequence known as LacO operator (LacO). By installing two copies of LacO on the expression plasmid, the LacI-peptide fusion binds tightly to the plasmid that encoded it. Because the plasmids in each cell contain only a single oligonucleotide sequence and each cell expresses only a single peptide sequence, the peptides become specifically and stably associated with the DNA sequence that directed its synthesis. The cells of the library are gently lysed and the peptide-DNA complexes are exposed to a matrix of immobilized receptor to recover the complexes containing active peptides. The associated plasmid DNA is then reintroduced into cells for amplification and DNA sequencing to determine the identity of the peptide ligands. As a demonstration of the practical utility of the method, a large random library of dodecapeptides was made and selected on a monoclonal antibody raised against the opioid peptide dynorphin B. A cohort of peptides was recovered, all related by a consensus sequence corresponding to a six-residue portion of dynorphin B. (Cull et al. (1992) Proc. Natl. Acad. Sci. U.S.A. 89-1869)

This scheme, sometimes referred to as peptides-on-plasmids, differs in two important ways from the phage display methods. First, the peptides are attached to the C-terminus of the fusion protein, resulting in the display of the library members as peptides having free carboxy termini. Both of the filamentous phage coat proteins, pIII and pVIII, are anchored to the phage through their C-termini, and the guest peptides are placed into the outward-extending N-terminal domains. In some designs, the phage-displayed peptides are presented right at the amino terminus of the fusion protein. (Cwirla, et al. (1990) Proc. Natl. Acad. Sci. U.S.A. 87, 6378-6382) A second difference is the set of biological biases affecting the population of peptides actually present in the libraries. The LacI fusion molecules are confined to the cytoplasm of the host cells. The phage coat fusions are exposed briefly to the cytoplasm during translation but are rapidly secreted through the inner membrane into the periplasmic compartment, remaining anchored in the membrane by their C-terminal hydrophobic domains, with the N-termini, containing the peptides, protruding into the periplasm while awaiting assembly into phage particles. The peptides in the LacI and phage libraries may differ significantly as a result of their exposure to different proteolytic activities. The phage coat proteins require transport across the inner membrane and signal peptidase processing as a prelude to incorporation into phage. Certain peptides exert a deleterious effect on these processes and are underrepresented in the libraries (Gallop et al. (1994) J. Med. Chem. 37(9):1233-1251). These particular biases are not a factor in the LacI display system.

The number of small peptides available in recombinant random libraries is enormous. Libraries of 10⁷-10⁹ independent clones are routinely prepared. Libraries as large as 10¹¹ recombinants have been created, but this size approaches the practical limit for clone libraries. This limitation in library size occurs at the step of transforming the DNA containing randomized segments into the host bacterial cells. To circumvent this limitation, an in vitro system based on the display of nascent peptides in polysome complexes has recently been developed. This display library method has the potential of producing libraries 3-6 orders of magnitude larger than the currently available phage/phagemid or plasmid libraries. Furthermore, the construction of the libraries, expression of the peptides, and screening, is done in an entirely cell-free format.

In one application of this method (Gallop et al. (1994) J. Med. Chem. 37(9):1233-1251), a molecular DNA library encoding 10¹² decapeptides was constructed and the library expressed in an E. coli S30 in vitro coupled transcription/translation system. Conditions were chosen to stall the ribosomes on the mRNA, causing the accumulation of a substantial proportion of the RNA in polysomes and yielding complexes containing nascent peptides still linked to their encoding RNA. The polysomes are sufficiently robust to be affinity purified on immobilized receptors in much the same way as the more conventional recombinant peptide display libraries are screened. RNA from the bound complexes is recovered, converted to cDNA, and amplified by PCR to produce a template for the next round of synthesis and screening. The polysome display method can be coupled to the phage display system. Following several rounds of screening, cDNA from the enriched pool of polysomes was cloned into a phagemid vector. This vector serves as both a peptide expression vector, displaying peptides fused to the coat proteins, and as a DNA sequencing vector for peptide identification. By expressing the polysome-derived peptides on phage, one can either continue the affinity selection procedure in this format or assay the peptides on individual clones for binding activity in a phage ELISA, or for binding specificity in a completion phage ELISA (Barret, et al. (1992) Anal. Biochem 204,357-364). To identify the sequences of the active peptides one sequences the DNA produced by the phagemid host.

Assays for IR Signaling Pathway Activity

The high through-put assays described above can be followed (or substituted) by secondary screens, e.g., the following screens, in order to identify biological activities which will, e.g., allow one skilled in the art to differentiate agonists from antagonists. The type of a secondary screen used will depend on the desired activity that needs to be tested. Several such assays are described below. For example, an assay can be developed in which the ability to inhibit an interaction between a protein of interest (e.g., IR) and a ligand (e.g., insulin or IRS) can be used to identify antagonists from a group of peptide fragments isolated though one of the primary screens described above.

Binding assays can be used to evaluate an IR signaling pathway activity. Component of the IR signaling pathway, e.g., insulin, IR, IRS, PI3K, AKT, PKC, SHC, SHP-2, GRB2, SOS-1 or Ras interact with each other, for example, to form active signaling or enzymatic complexes. For example, insulin binds IR, which causes activation of the IR signaling pathway; IR binds and phosphorylates IRS. Thus, the ability of one component to bind a binding partner is an assayable activity of the IR signaling pathway. Thus, a binding assay, e.g., a binding assay described herein, can be used to evaluate: (a) the ability of a test agent to bind a component of the IR signaling pathway, e.g., insulin, IR, IRS, PI3K, AKT, PKC, SHC, SHP-2, GRB2, SOS-1 or Ras; (b) the ability of a test agent to inhibit binding of component to a binding partner, e.g., the ability of a test agent to inhibit or disrupt insulin binding to IR or IR binding to IRS; (c) the ability of a test agent to stabilize or increase binding of a component to a binding partner, e.g., the ability of a test agent to stabilize or increase insulin binding to IR or IR binding to IRS.

As most components of the IR signaling pathway, e.g., insulin, IR, IRS, P13K, AKT, PKC, SHC, SHP-2, GRB2, SOS-1 or Ras can be purified, e.g., from mammals and/or have been cloned and produced recombinantly, they are readily available as reagents to be used in standard binding assays known in the art, which include, but are not limited to: affinity chromatography, size exclusion chromatography, gel filtration, fluid phase binding assay; ELISA (e.g., competition ELISA), immunoprecipitation. Such techniques are well known in the art.

IR signaling pathway activity can also be evaluated by measuring an enzymatic activity of the alternative pathway, e.g., by measuring IR tyrosine kinase activity. For example, IR tyrosine kinase activity can be assayed by evaluating the extent of IRS phosphorylation, e.g., in vitro, or in an adipose cell. Standard kinase assays can be used for this purpose.

Administration

An agent that modulates the IR signaling pathway, e.g., an agent that inhibits insulin, IR, IRS, PI3K, AKT, PKC, SHC, SHP-2, GRB2, SOS-1 or Ras, e.g., an agent described herein, can be administered to a subject by standard methods. For example, the agent can be administered by any of a number of different routes including intravenous, intradermal, subcutaneous, oral (e.g., inhalation), transdermal (topical), and transmucosal. In one embodiment, the modulating agent can be administered orally. In another embodiment, the agent is administered by injection, e.g., intramuscularly, or intravenously. In preferred embodiments, the agent is targeted, e.g., includes a targeting reagent, to an adipocyte tissue.

Any agent that modulates the IR signaling pathway, e.g., reduces IR signaling, e.g., an agent described herein, e.g., nucleic acid molecules, polypeptides, fragments or analogs, modulators, organic compounds and antibodies (also referred to herein as “active compounds”) can be incorporated into pharmaceutical compositions suitable for administration to a subject, e.g., a human. Such compositions typically include the nucleic acid molecule, polypeptide, modulator, or antibody and a pharmaceutically acceptable carrier. As used herein the language “pharmaceutically acceptable carrier” is intended to include any and all solvents, dispersion media, coatings, antibacterial and antifingal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions.

A pharmaceutical composition can be formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an agent described herein) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

The nucleic acid molecules described herein can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al., PNAS 91:3054-3057, 1994). The pharmaceutical preparation of the gene therapy vector can include the gene therapy vector in an acceptable diluent, or can include a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

In a preferred embodiment, the pharmaceutical composition is administered directly into an adipose tissue of the subject.

Gene Therapy

The nucleic acids described herein, e.g., an antisense nucleic acid described herein, can be incorporated into gene constructs to be used as a part of a gene therapy protocol to deliver nucleic acids encoding either an agonistic or antagonistic form of an IR signaling pathway component described herein. The invention features expression vectors for in vivo transfection and expression of an alternative pathway component described herein in particular cell types so as to reconstitute the function of, or alternatively, antagonize the function of the component in a cell in which that polypeptide is misexpressed. Expression constructs of such components may be administered in any biologically effective carrier, e.g. any formulation or composition capable of effectively delivering the component gene to cells, preferably adipose cells, in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO4 precipitation carried out in vivo.

A preferred approach for in vivo introduction of nucleic acid into a cell is by use of a viral vector containing nucleic acid, e.g. a cDNA, encoding an IR signaling pathway component described herein. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, A. D. (1990) Blood 76:271). A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include *Crip, *Cre, *2 and *Am. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 d1324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267).

Yet another viral vector system useful for delivery of the subject gene is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. (1992) Curr. Topics in Micro. and Immunol.158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hernonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

In addition to viral transfer methods, such as those illustrated above, non-viral methods can also be employed to cause expression of an IR signaling pathway component described herein in the tissue of a subject. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al. (2001) J Invest Dermatol. 116(1):131-135; Cohen et al. (2000) Gene Ther 7(22):1896-905; or Tam et al. (2000) Gene Ther 7(21):1867-74.

In a representative embodiment, a gene encoding an IR signaling pathway component described herein can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication WO91/06309; Japanese patent application 1047381; and European patent publication EP-A-43075).

In clinical settings, the gene delivery systems for the therapeutic gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) PNAS 91: 3054-3057).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced in tact from recombinant cells, e.g. retroviral vectors, the pharmaceutical preparation can comprise one or more cells which produce the gene delivery system.

Cell Therapy

An IR signaling pathway component described herein, e.g., insulin, IR, IRS, PI3K, AKT, PKC, SHC, SHP-2, GRB2, SOS-1 or Ras, can also be increased in a subject by introducing into a cell, e.g., an adipocyte, a nucleotide sequence that modulates the production of an IR signaling pathway component described herein, e.g., a nucleotide sequence encoding an IR signaling pathway component described herein, polypeptide or functional fragment or analog thereof, a promoter sequence, e.g., a promoter sequence from an IR signaling pathway component gene or from another gene; an enhancer sequence, e.g., 5′ untranslated region (UTR), e.g., a 5′ UTR from an IR signaling pathway component gene or from another gene, a 3′ UTR, e.g., a 3′ UTR from an IR signaling pathway component gene or from another gene,; a polyadenylation site; an insulator sequence; or another sequence that modulates the expression of the IR signaling pathway component. The cell can then be introduced into the subject.

Primary and secondary cells to be genetically engineered can be obtained form a variety of tissues and include cell types which can be maintained propagated in culture. For example, primary and secondary cells include fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells (myoblasts) and precursors of these somatic cell types. Primary cells are preferably obtained from the individual to whom the genetically engineered primary or secondary cells are administered. However, primary cells may be obtained for a donor (other than the recipient). Preferred cells are adipocytes, e.g., WAT adipocytes.

The term “primary cell” includes cells present in a suspension of cells isolated from a vertebrate tissue source (prior to their being plated i.e., attached to a tissue culture substrate such as a dish or flask), cells present in an explant derived from tissue, both of the previous types of cells plated for the first time, and cell suspensions derived from these plated cells. The term “secondary cell” or “cell strain” refers to cells at all subsequent steps in culturing. Secondary cells are cell strains which consist of secondary cells which have been passaged one or more times.

Primary or secondary cells of vertebrate, particularly mammalian, origin can be transfected with an exogenous nucleic acid sequence which includes a nucleic acid sequence encoding a signal peptide, and/or a heterologous nucleic acid sequence, e.g., encoding an IR signaling pathway component, or an agonist or antagonist thereof, and produce the encoded product stably and reproducibly in vitro and in vivo, over extended periods of time. A heterologous amino acid can also be a regulatory sequence, e.g., a promoter, which causes expression, e.g., inducible expression or upregulation, of an endogenous sequence. An exogenous nucleic acid sequence can be introduced into a primary or secondary cell by homologous recombination as described, for example, in U.S. Pat. No. 5,641,670, the contents of which are incorporated herein by reference. The transfected primary or secondary cells may also include DNA encoding a selectable marker which confers a selectable phenotype upon them, facilitating their identification and isolation.

Vertebrate tissue can be obtained by standard methods such a punch biopsy or other surgical methods of obtaining a tissue source of the primary cell type of interest. For example, punch biopsy is used to obtain skin as a source of fibroblasts or keratinocytes. A mixture of primary cells is obtained from the tissue, using known methods, such as enzymatic digestion or explanting. If enzymatic digestion is used, enzymes such as collagenase, hyaluronidase, dispase, pronase, trypsin, elastase and chymotrypsin can be used.

The resulting primary cell mixture can be transfected directly or it can be cultured first, removed from the culture plate and resuspended before transfection is carried out. Primary cells or secondary cells are combined with exogenous nucleic acid sequence to, e.g., stably integrate into their genomes, and treated in order to accomplish transfection. As used herein, the term “transfection” includes a variety of techniques for introducing an exogenous nucleic acid into a cell including calcium phosphate or calcium chloride precipitation, microinjection, DEAE-dextrin-mediated transfection, lipofection or electrophoration, all of which are routine in the art.

Transfected primary or secondary cells undergo sufficient number doubling to produce either a clonal cell strain or a heterogeneous cell strain of sufficient size to provide the therapeutic protein to an individual in effective amounts. The number of required cells in a transfected clonal heterogeneous cell strain is variable and depends on a variety of factors, including but not limited to, the use of the transfected cells, the functional level of the exogenous DNA in the transfected cells, the site of implantation of the transfected cells (for example, the number of cells that can be used is limited by the anatomical site of implantation), and the age, surface area, and clinical condition of the patient.

The transfected cells, e.g., cells produced as described herein, can be introduced into an individual to whom the product is to be delivered. Various routes of administration and various sites (e.g., renal sub capsular, subcutaneous, central nervous system (including intrathecal), intravascular, intrahepatic, intrasplanchnic, intraperitoneal (including intraomental), intramuscularly implantation) can be used. One implanted in individual, the transfected cells produce the product encoded by the heterologous DNA or are affected by the heterologous DNA itself. For example, an individual who suffers from an antibody-mediated arthritic disorder is a candidate for implantation of cells producing an antagonist of the alternative pathway described herein.

An immunosuppressive agent e.g., drug, or antibody, can be administered to a subject at a dosage sufficient to achieve the desired therapeutic effect (e.g., inhibition of rejection of the cells). Dosage ranges for immunosuppressive drugs are known in the art. See, e.g., Freed et al. (1992) N. Engl. J. Med. 327:1549; Spencer et al. (1992) N. Engl. J. Med. 327:1541′ Widner et al. (1992) n. Engl. J. Med. 327:1556). Dosage values may vary according to factors such as the disease state, age, sex, and weight of the individual.

Predictive Medicine

The present invention also pertains to the field of predictive medicine in which diagnostic assays, prognostic assays, and monitoring clinical trials are used for prognostic (predictive) purposes to thereby treat an individual.

Generally, the invention provides, a method of determining if a subject is at risk for diabetes or a diabetes related disorder.

Such disorders include, e.g., hypertension and retinopathy, persistent hyperinsulinemic hypoglycaemia of infancy (PHHI), insulin resistance, hyperglycemia, glucose intolerance, glucotoxicity, or β-cell dysfunction.

The method includes one or more of the following:
detecting, in a tissue of the subject, the presence or absence of a mutation which affects the expression of a marker described herein, or detecting the presence or absence of a mutation in a region which controls the expression of the marker gene, e.g., a mutation in the 5′ control region, intron, or 3′ untranslated region;
detecting, in a tissue of the subject, the presence or absence of a mutation which alters the structure of the marker gene;
detecting, in a tissue of the subject, the misexpression of the marker gene, at the mRNA level, e.g., detecting a non-wild type level of a mRNA;
detecting, in a tissue of the subject, the misexpression of the marker, at the protein level, e.g., detecting a non-wild type level of a marker polypeptide.

In preferred embodiments the method includes: ascertaining the existence of at least one of: a deletion of one or more nucleotides from the marker gene; an insertion of one or more nucleotides into the gene, a point mutation, e.g., a substitution of one or more nucleotides of the gene, a gross chromosomal rearrangement of the gene, e.g., a translocation, inversion, or deletion.

For example, detecting the genetic lesion can include: (i) providing a probe/primer including an oligonucleotide containing a region of nucleotide sequence which hybridizes to a sense or antisense sequence from a sequence of the marker gene, or naturally occurring mutants thereof or 5′ or 3′ flanking sequences naturally associated with the gene; (ii) exposing the probe/primer to nucleic acid of the tissue; and detecting, by hybridization, e.g., in situ hybridization, of the probe/primer to the nucleic acid, the presence or absence of the genetic lesion.

In preferred embodiments detecting the misexpression includes ascertaining the existence of at least one of: an alteration in the level of a messenger RNA tran; the presence of a non-wild type splicing pattern of a messenger RNA transcript; or a non-wild type level of marker protein.

Methods of the invention can be used prenatally or to determine if a subject's offspring will be at risk for a disorder.

In preferred embodiments the method includes determining the structure of a marker gene (e.g., a marker described herein), an abnormal structure being indicative of risk for the disorder.

In preferred embodiments the method includes contacting a sample from the subject with an antibody to the marker protein; or a nucleic acid, which hybridizes specifically with the gene. These and other embodiments are discussed below.

Diagnostic and Prognostic Assays

Diagnostic and prognostic assays of the invention include method for assessing the expression level of marker molecules and for identifying variations and mutations in the sequence of marker molecules, e.g., markers described herein.

Expression Monitoring and Profiling. The presence, level, or absence of a marker protein or nucleic acid in a biological sample can be evaluated by obtaining a biological sample from a test subject and contacting the biological sample with a compound or an agent capable of detecting the marker protein or nucleic acid (e.g., mRNA, genomic DNA) that encodes the protein such that the presence of the protein or nucleic acid is detected in the biological sample. The term “biological sample” includes tissues, cells and biological fluids isolated from a subject, as well as tissues, cells and fluids present within a subject. A preferred biological sample is serum. The level of expression of the marker gene can be measured in a number of ways, including, but not limited to: measuring the mRNA encoded by the genes; measuring the amount of protein encoded by the genes; or measuring the activity of the protein encoded by the genes.

The level of mRNA corresponding to the gene in a cell can be determined both by in situ and by in vitro formats.

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length nucleic acid, or a portion thereof, such as an oligonucleotide of at least 7, 15, 30, 50, 100, 250 or 500 nucleotides in length and sufficient to specifically hybridize under stringent conditions to the mRNA corresponding to the marker gene or genomic DNA. The probe can be disposed on an address of an array, e.g., an array described below. Other suitable probes for use in the diagnostic assays are described herein.

In one format, mRNA (or cDNA) is immobilized on a surface and contacted with the probes, for example by running the isolated mRNA on an agarose gel and transferring the mRNA from the gel to a membrane, such as nitrocellulose. In an alternative format, the probes are immobilized on a surface and the mRNA (or cDNA) is contacted with the probes, for example, in a two-dimensional gene chip array described below. A skilled artisan can adapt known mRNA detection methods for use in detecting the level of mRNA encoded by the marker gene.

The level of mRNA in a sample that is encoded by a marker, e.g., a marker described herein, can be evaluated with nucleic acid amplification, e.g., by rtPCR (Mullis (1987) U.S. Pat. No. 4,683,202), ligase chain reaction (Barany (1991) Proc. Natl. Acad. Sci. USA 88:189-193), self sustained sequence replication (Guatelli et al., (1990) Proc. Natl. Acad. Sci. USA 87:1874-1878), transcriptional amplification system (Kwoh et al., (1989), Proc. Natl. Acad. Sci. USA 86:1173-1177), Q-Beta Replicase (Lizardi et al., (1988) Bio/Technology 6:1197), rolling circle replication (Lizardi et al., U.S. Pat. No. 5,854,033) or any other nucleic acid amplification method, followed by the detection of the amplified molecules using techniques known in the art. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, a cell or tissue sample can be prepared/processed and immobilized on a support, typically a glass slide, and then contacted with a probe that can hybridize to mRNA that encodes the gene being analyzed.

In another embodiment, the methods further include contacting a control sample with a compound or agent capable of detecting mRNA, or genomic DNA, and comparing the presence of mRNA or genomic DNA in the control sample with the presence of mRNA or genomic DNA in the test sample. In still another embodiment, serial analysis of gene expression, as described in U.S. Pat. No. 5,695,937, is used to detect transcript levels.

A variety of methods can be used to determine the level of protein encoded by a marker, e.g., a marker described herein. In general, these methods include contacting an agent that selectively binds to the protein, such as an antibody with a sample, to evaluate the level of protein in the sample. In a preferred embodiment, the antibody bears a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with a detectable substance. Examples of detectable substances are provided herein.

The detection methods can be used to detect protein in a biological sample in vitro as well as in vivo. In vitro techniques for detection of protein include enzyme linked immunosorbent assays (ELISAs), immunoprecipitations, immunofluorescence, enzyme immunoassay (EIA), radioimmunoassay (RIA), and Western blot analysis. In vivo techniques for detection of protein include introducing into a subject a labeled anti-marker antibody. For example, the antibody can be labeled with a radioactive marker whose presence and location in a subject can be detected by standard imaging techniques. In another embodiment, the sample is labeled, e.g., biotinylated and then contacted to the antibody, e.g., an anti-antibody positioned on an antibody array (as described below). The sample can be detected, e.g., with avidin coupled to a fluorescent label.

In another embodiment, the methods further include contacting the control sample with a compound or agent capable of detecting the marker protein, and comparing the presence of protein in the control sample with the presence of protein in the test sample.

This invention is further illustrated by the following examples that should not be construed as limiting. The contents of all references, patents and published patent applications cited throughout this application are incorporated herein by reference.

### EXAMPLES

### Example 1

### Creation and Molecular Characterization of the Fat-Specific IR Knockout Mice

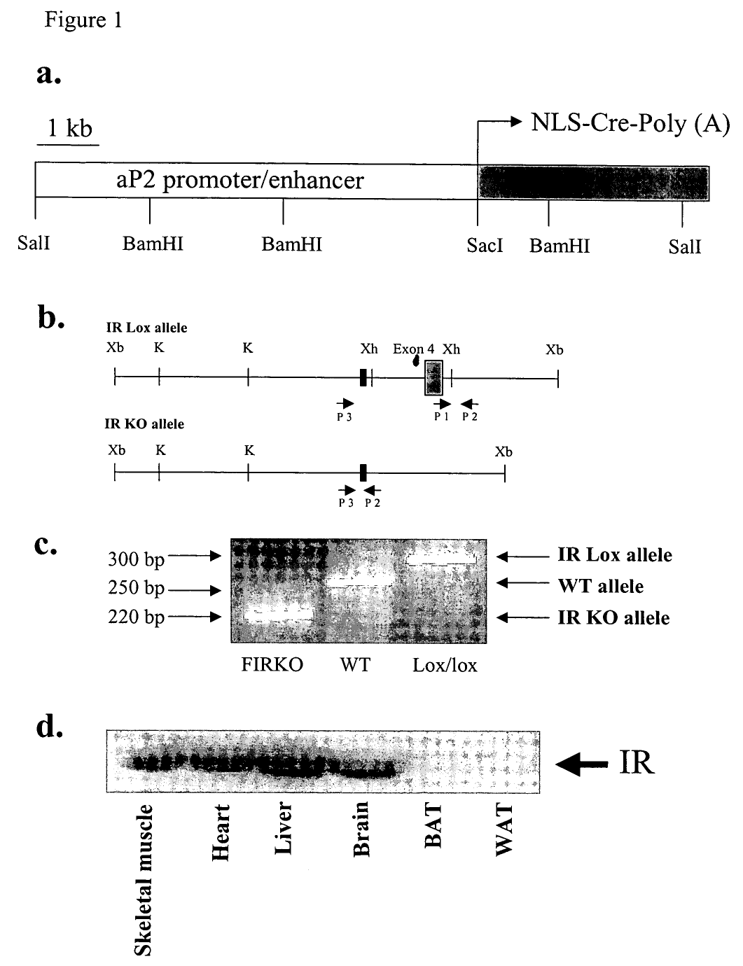

Fat-specific insulin receptor knockout (FIRKO) mice were generated by breeding IR (lox/+) mice (Brüning et al., 1998) with transgenic mice that express the Cre recombinase cDNA from the adipose specific fatty-acid-binding protein (aP2) promoter/enhancer (Ross et al., 1990) (FIG. 1a). FIRKO mice were obtained with the expected Mendelian frequency and exhibited normal growth until the age of 8 weeks. Cre expression was restricted to white adipose tissue (WAT) and brown adipose tissue (BAT).

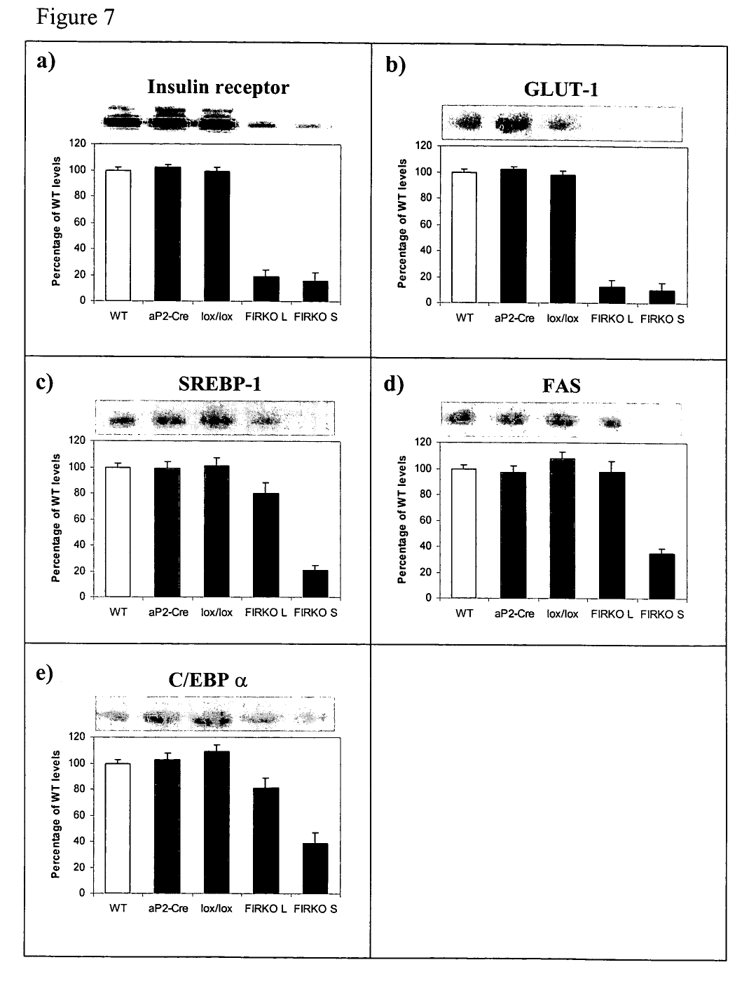

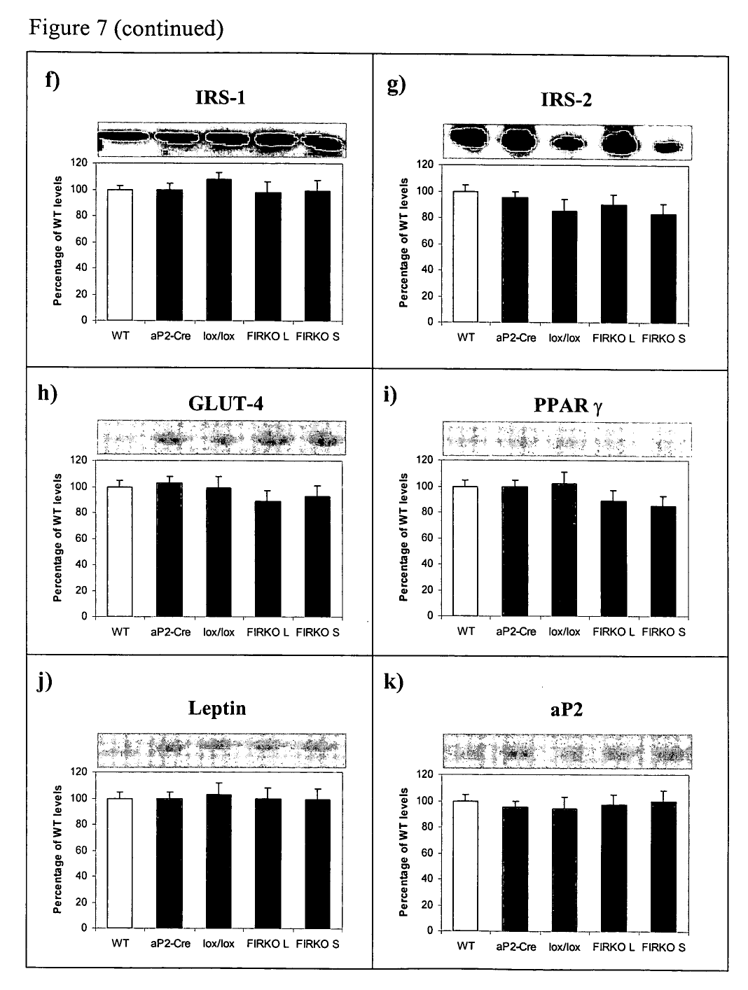

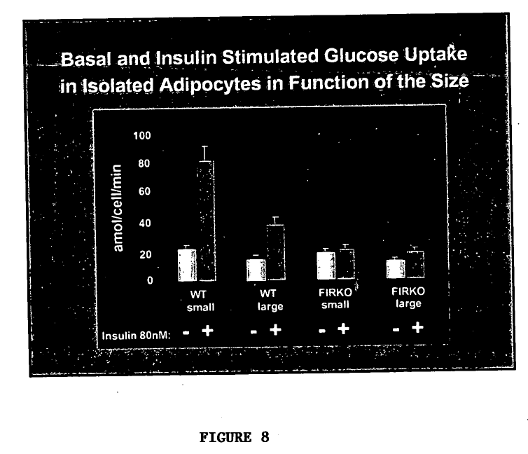

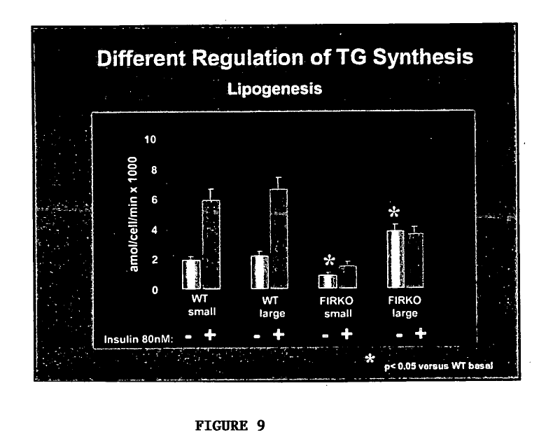

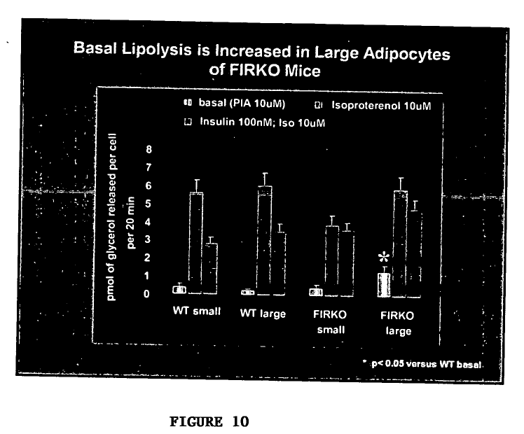

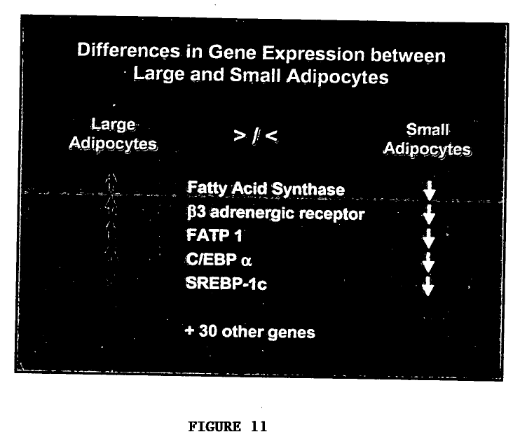

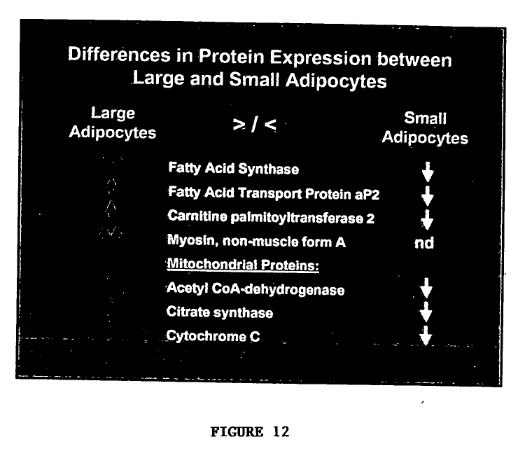

Efficiency and specifity of the IR knockout were examined in isolated adipocytes and tissue lysates from control and FIRKO mice by immunoprecipitation with an IR-specific antiserum followed by Western blot analysis with the same antiserum. The IR expression was preserved in skeletal muscle, liver, brain, heart and other tissues examined (FIG. 1d). IR expression was unaffected in isolated adipocytes in the brown (data not shown) and white adipose tissue of WT, IR (lox/lox), and aP2-Cre mice (FIG. 7a) indicating that neither the loxP modification of the IR locus nor expression of the aP2 transgene alone affects IR expression. These control genotypes WT, IR (lox/lox), and aP2-Cre had similar physiologic and metabolic characteristics, and were considered controls. IR protein expression was reduced by 85-99% in isolated adipocytes of FIRKO mice. The remaining IR expression could either be derived from vascular endothelial cells or stromal cells contaminating the isolated adipocytes or be related to adipocytes, which escape aP2 expression. To assure uniformity of the FIRKO study groups, IR recombination was assessed in WAT of each mouse (FIG. 1c), and only data from mice with an efficient IR recombination were included in the analysis. The tissue specificity and high efficiency of Cre activity were consistent with previous studies in which the aP2-Cre mice were crossed with the ROSA26-lacZ reporter mouse (Abel et al., 2001, Zambrowicz et al., 1997).

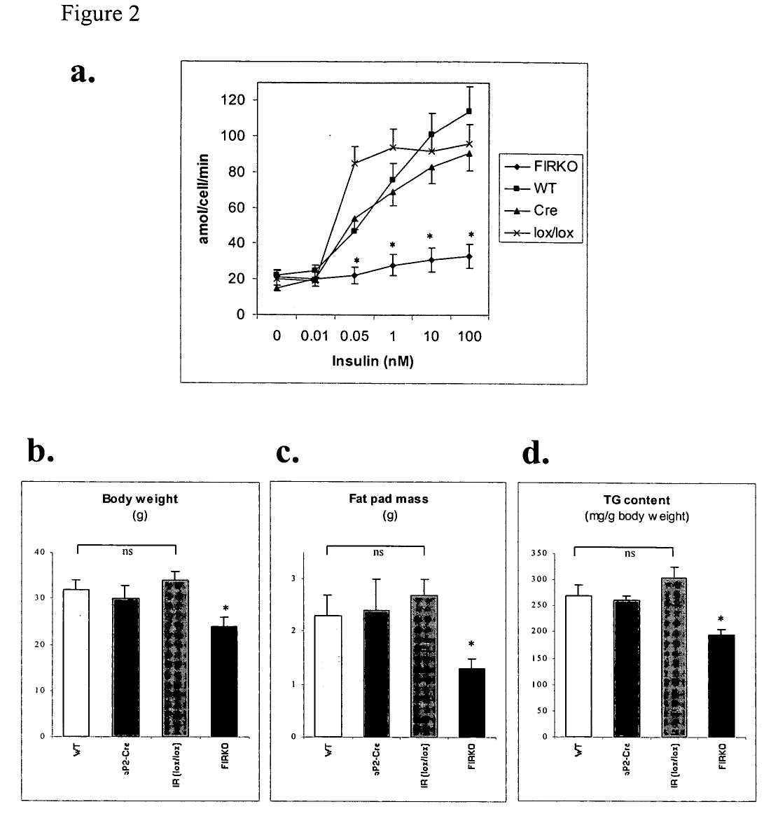

To determine the consequence of reduced IR-mediated signaling, basal and insulin-stimulated glucose transport in isolated adipocytes from FIRKO mice and control littermates was studied. In adipocytes from FIRKO mice, basal glucose uptake is unchanged compared to the controls, but insulin-stimulated glucose uptake is reduced by ˜90% at all insulin concentrations from 0.05 nM to 100 nM (FIG. 2a). The observed insulin resistance in FIRKO adipocytes confirms the efficiency of the adipocyte-specific IR knockout and is similar to that in mice with homozygous gene knockout of the insulin-sensitive glucose transporter GLUT4 (Abel et al., 2001).

### Example 2

### Physiological Consequence of Fat-Specific IR Knockout

Body Fat is Markedly Reduced in FIRKO Mice

Growth curves were normal in male and female FIRKO mice from birth to four weeks of age. By 8 weeks of age, however, FIRKO mice had gained less weight than control group littermates (FIG. 2b). In addition, perigonadal fat pad mass (FIG. 2c), intrascapular brown fat pad mass (2.77±0.15 mg/g body weight in the controls versus 1.21±0.12 mg/g body weight in FIRKO mice at the age of 3 months) and whole body triglyceride content was significantly lower in FIRKO mice compared to the control groups (FIG. 2d). The reduced adipose tissue mass was not related to a decrease of the total number of adipocytes in FIRKO mice. The number of adipocytes per perigonadal fat pad was not significantly different between FIRKO (4.13±0.18×10⁶ cells) and control (3.97±0.24×10⁶ cells) mice. Despite the >50% reduction in BAT mass, the expression of UCP-1, at both the mRNA and protein level was indistinguishable between BAT from FIRKO mice and controls; when expressed per mg of BAT mass, UCP-1 expression (both mRNA and protein) was increased in BAT of FIRKO mice.

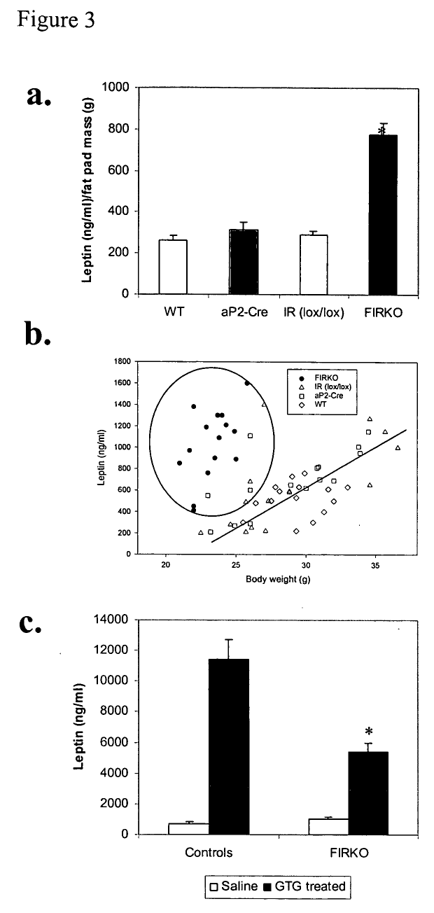

Despite the decreased whole body fat mass, FIRKO mice of both genders had about 25% higher plasma leptin levels than control groups, although this difference was not statistically significant (Table 1). However, when expressed per mg of fat pad mass, plasma leptin levels in FIRKO mice were ˜3 fold elevated (FIG. 3a, b), and the linear relationship between leptin levels and body weight seen in the control groups was lost (FIG. 3b), suggesting that adipose specific IR knockout causes alterations in the leptin regulation.

Metabolic Parameters

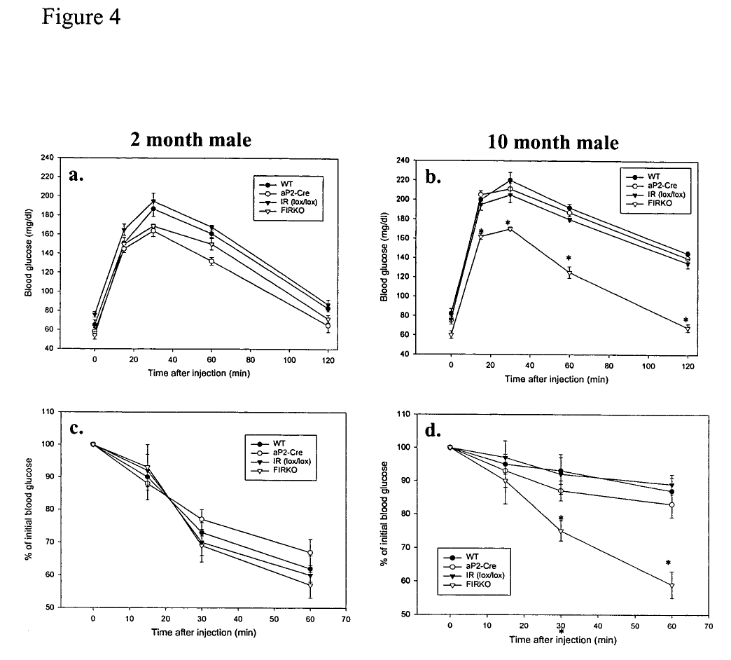

To determine the physiological consequences of the fat-specific IR knockout, body weight, blood glucose concentration and insulin levels were monitored in the fasted and fed state, and triglycerides, cholesterol, free fatty acids (FFA), and leptin in plasma and serial glucose insulin tolerance testing was performed over an age range from 2 to 10 months. Fasted and fed glucose concentrations were indistinguishable between FIRKO mice and control littermates at 2-8 months (Table 1). Although there was no significant difference in the plasma fed insulin concentrations, FIRKO mice showed significantly lower fasted insulin concentrations compared to WT and aP2-Cre mice (p<0.05) (Table 1). Serum triglyceride levels were significantly reduced in FIRKO mice compared to WT and IR (lox/lox) mice (Table 1), whereas serum FFA, plasma leptin (Table 1) and cholesterol (Table 1) as well as lactate levels were not significantly different among the groups. Likewise, intraperitoneal glucose tolerance testing (GTT) performed on 2-month-old, male FIRKO and control mice demonstrated normal glucose tolerance in all groups (FIG. 4a). However, by the age of 10 months, all control groups showed impaired glucose tolerance due to increasing insulin resistance associated with aging, whereas FIRKO mice maintained normal glucose tolerance (FIG. 4b). Intraperitoneal insulin tolerance tests (ITT) at 2 months of age in male mice were indistinguishable between FIRKO and control mice (FIG. 4c). Insulin resistance increased by 10 months of age in all control groups, but not in FIRKO mice (FIG. 4d).

TABLE 1
METABOLIC PARAMETERS IN 2 MONTHS OLD
MALE FIRKO AND CONTROL MICE

| | WT | aP2-Cre | IR(lox/lox) | FIRKO |
|---|---|---|---|---|
| Fasted Glucose | 56 ± 2 | 54 ± 3 | 58 ± 5 | 57 ± 6 |
| (mg/dl) | | | | |
| Fasted Insulin | 260 ± 39 | 232 ± 30 | 222 ± 66 | 151 ± 22 |
| (pg/ml) | | | | |
| Fed Glucose | 147 ± 3 | 148 ± 11 | 135 ± 7 | 141 ± 9 |
| (mg/dl) | | | | |
| Fed Insulin | 1367 ± 239 | 1334 ± 202 | 1265 ± 150 | 1349 ± 219 |
| (pg/ml) | | | | |
| Triglycerides | 170 ± 26 | 142 ± 13 | 177 ± 28 | 129 ± 19 |
| (mg/dl) | | | | |
| Cholesterol | 131 ± 28 | 127 ± 18 | 119 ± 22 | 108 ± 17 |
| (mg/dl) | | | | |
| FFAs (mEq/L) | 1183 ± 89 | 1278 ± 83 | 1157 ± 114 | 1054 ± 145 |
| Leptin (pg/ml) | 577 ± 163 | 723 ± 167 | 811 ± 232 | 1010 ± 360 |

*indicates significant difference from WT and aP2-Cre mice,
+ indicates significance differences from WT and IR (lox/lox). (p < 0.05)

### Example 3

### FIRKO Mice are Protected from Goldthioglucose Induced Obesity and Glucose Intolerance

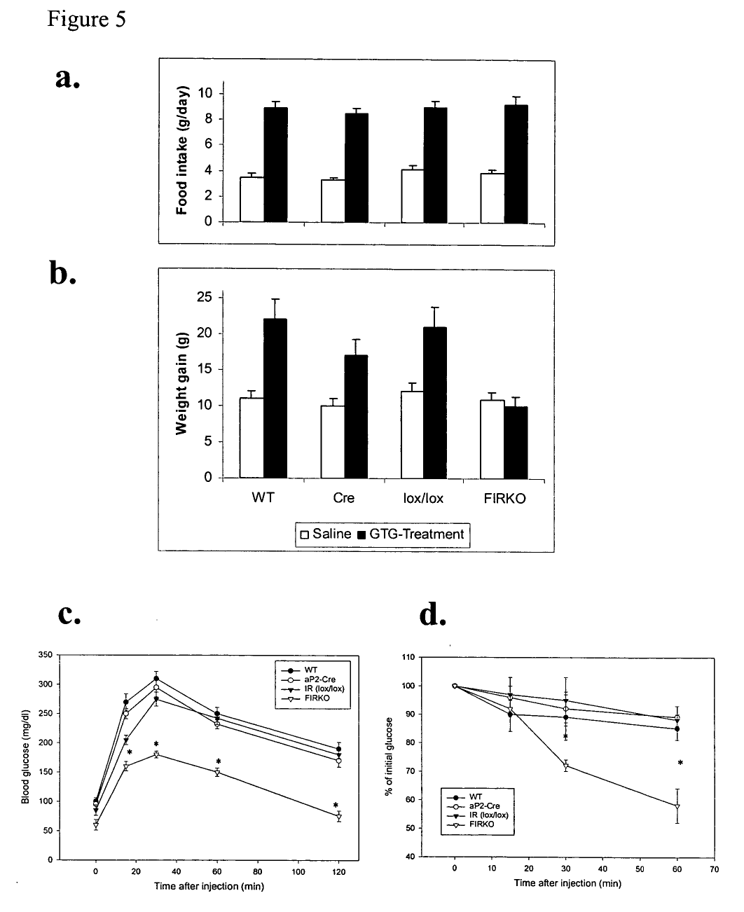

Gold thioglucose (GTG) treatment results in specific lesions in the ventromedial hypothalamus with subsequent development of hyperphagia and obesity (Debons et al., 1977). To assess the impact of this hyperphagia in this model, 4 week old FIRKO mice and their littermates were treated with either 0.5 mg/g body weight GTG or normal saline (control group), and body weight and food intake were obtained before and 12 weeks after treatment. In both FIRKO and control mice, daily food intake increased ˜2-3 fold after GTG treatment as compared to saline treated mice (FIG. 5a). As a result, there was a 60-100% increase of weight gain and in the development of obesity in WT, IR (lox/lox), and aP2-Cre mice. Remarkably, despite the hyperphagia, FIRKO mice treated with GTG, had weight gain comparable to that observed in their saline treated littermates (FIG. 5b). Serum leptin levels increased in all GTG-treated mice, but were significantly lower in the GTG-treated FIRKOs as compared to the GTG-treated controls (FIG. 3c). Moreover, intraperitoneal glucose tolerance testing performed 12 weeks after GTG treatment, demonstrated normal glucose tolerance in FIRKO mice, whereas all of the control groups had developed significantly impaired glucose tolerance (FIG. 5c). Insulin sensitivity, as determined by insulin tolerance testing, also remained normal in FIRKO mice after GTG treatment, whereas WT, IR (lox/lox), and aP2-Cre mice displayed marked insulin resistance (FIG. 5d). Thus, the adipose specific IR knockout in FIRKO mice protects from GTG-induced, as well as from age-related, obesity and obesity-related glucose intolerance and insulin resistance.

### Example 4

### IR Knockout in Adipose Tissue Causes a Polarization in the Adipocyte Size with Differences in the Protein Expression

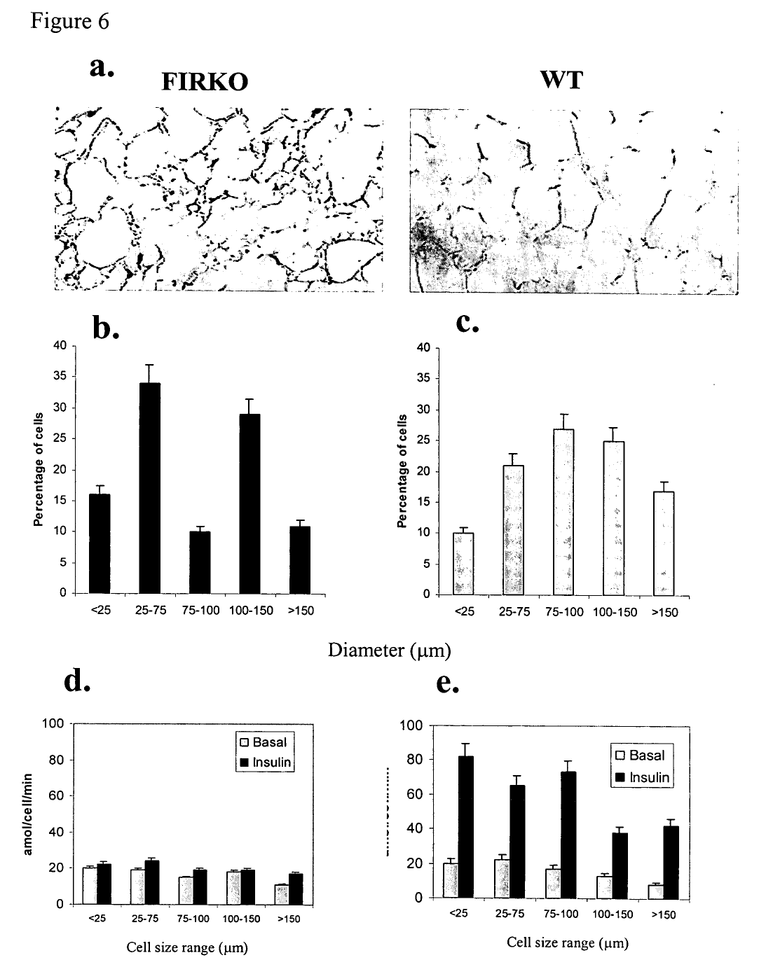

To evaluate the impact of loss of the IR on adipose tissue morphology, histological studies on the WAT of FIRKO and control mice were performed. At 2 months of age, fat pads from FIRKO mice contained a mixed population of large and small adipocytes as compared to the relatively uniform adipocyte size in WAT from WT, IR (lox/lox), and aP2-Cre mice (FIG. 6a). Quantitation of these histologic sections revealed a polarization of adipocytes into two major groups in FIRKO mice: small cells with a diameter <75 μm and large cells with a diameter >100 μm with only 7.6±1.3% of the in the size range of 75-100 μm (FIG. 6c). For WT mice, there was a normal distribution of cell size with the major fraction (26.7±2.8%) being in the range of 75-100 μm (FIG. 6b). This polarization of cell size was confirmed by FACS analysis of osmic acid fixed isolated adipocytes, which revealed a significant increase in the percentage of small adipocytes, i.e., cells with a diameter less than 75 μm, in FIRKO mice (46.4±4.3% of total cell number) as compared to those in fat pads of WT mice (29.8±2.6% of total cell number) (p<0.05).

To further characterize these different sized adipocytes, cells were fractionated by filtering the adipocyte suspension through nylon mesh screens of different pore size, and analyzed with respect to glucose uptake and expression of several key regulatory proteins. As compared to controls, IR expression in both large and small adipocytes of FIRKO mice was reduced by 85-99%, indicating that the heterogeneity was not due to differences in efficiency of gene recombination in the small and large cells (see FIG. 7a). This was confirmed by PCR analysis of small and large adipocytes of FIRKO mice. Basal glucose uptake in WT adipocytes decreased slightly with increasing adipocyte size, and became significant in adipocytes with a diameter >150 μm. As previously observed (Foley et al., 1980), smaller adipocytes (diameter <100 μm) from control mice were also significantly more responsive to insulin than large adipocytes (diameter >100 μm) in terms of insulin-stimulated glucose uptake (FIG. 6e). In FIRKO mice, basal glucose uptake in adipocytes was not different among the cell size fractions (FIG. 6d), and there was a lack of insulin stimulated glucose transport in any cell size range, confirming the insulin receptor was knocked out in all adipose cell size groups.

To examine some potential differences between the small (<75 μm) and large (>75 μm) adipocytes from FIRKO mice, the expression of several key adipocyte proteins that might be regulated in response to the IR knockout was measured. Three different patterns of expression were observed: 1) decreased levels in both large and small FIRKO adipocytes as compared to controls; 2) differential levels in large and small FIRKO adipocytes; 3) unchanged levels in FIRKO cells as compared to the control groups. The first pattern, i.e., decreased levels in both large and small FIRKO cells, was observed for the insulin receptor (FIG. 7a) and the GLUT1 glucose transporter (FIG. 7b). The former was expected based on the knockout efficiency; the latter showed normal that insulin action is crucial for GLUT1 protein expression in vivo. The second pattern of expression with differential expression between large and small cells was observed for the adipogenic transcription factors SREBP-1 (FIG. 7c) and C/EBPα (FIG. 7e), both of which were reduced in FIRKO adipocytes of both size groups as compared to adipocytes from the control mice, but were more markedly decreased in FIRKO small adipocytes compared to FIRKO large adipocytes. This differential pattern of expression was also observed for the levels of fatty acid synthase (FAS), however, in this case, levels in large cells were indistinguishable from those in controls, whereas small adipocytes from the FIRKO mice had significantly reduced expression (FIG. 7d). The final pattern of expression, i.e., no change in amount in either large or small FIRKO adipocytes, was observed for the GLUT4 glucose transporter (FIG. 7h), the adipogenic transcription factor PPARγ (FIG. 7i), the fatty acid binding protein aP2 (FIG. 7k), leptin protein levels (FIG. 7j), and the insulin receptor substrates IRS-1 and IRS-2 (FIG. 7f, g). There was also no significant difference in the levels of any of the analyzed proteins between small and large adipocyte fractions from the three control groups WT, IR (lox/lox), and aP2-Cre mice.

### Example 5

### Different Fat Depots Have Different Genie Expression Profiles

The white adipose in visceral depots appears to be different from that in subcutaneous depots such that individuals with peripheral obesity are at low risk of the common medical complications of obesity, whereas individuals with central obesity (i.e. fat accumulation in visceral depots) are prone to atherosclerosis, dyslipidemia, hypertension and diabetes. Acylation stimulating protein and angiotensinogen mRNA levels are higher in visceral adipose tissue, whereas the mRNA levels of leptin, PPAR-gamma, GLUT4, glycogen synthase and cholesterol ester transfer protein are higher in the subcutaneous depot. To what extent these interdepot variations result from extrinsic influences (like hormonal and paracrine microenvironement) versus intrinsic properties of the adipocytes in these different depots remains unknown.

Methods

In order to begin to define the genes differentially expressed in the different adipose tissue depots, in this Example we isolated adipocytes by collagenase digestion and floatation (to avoid contaminating cells) from three different fat depots (epididymal, visceral and subcutaneous) of C57BL/6 mice and performed gene expression analysis. 25 μg of RNA was extracted from isolated adipocytes and used to create 15 μg of biotinylated cRNA for chip analysis. Due to the low yield of RNA, adipocytes RNA from 10 mice was pooled for each chip. Three sequential statistical filters were used to determine genes that were differentially expressed. The first filter excluded genes that had a mean expression value below the sum of the average background and the average standard difference threshold (SDT, equal to four times the scaled noise). Genes that passed this filter were subjected to the second filter, which was to consider only those genes for which the absolute difference between the means of the groups was greater than the average SDT. Finally, the third filter of significance was to select those genes that had passed both of the above filters and for which the difference between the means of the groups was ≧2 times the sum of the standard deviations of both groups This experiment was repeated 3 times. Results are summarized in Table 2 below.

Results

553 genes and ESTs (386 known and 167 unknown) could be defined as differentially expressed between adipocytes from epididymal and subcutaneous adipose tissue. In contrast to some of the other disease states studied, many of these differences were quite substantial. Thus, 72 genes were expressed at levels at least 2 fold higher in epididymal than subcutaneous adipocytes, and 89 were at least 2-fold higher in subcutaneous as compared to epididymal. Among the former, were mRNAs for cytochrome p450 1b1, an enzyme that metabolizes polycyclic aromatic hydrocarbons such as 17-beta-estradiol (11-fold higher in epididymal fat) and angiotensinogen (7.5-fold higher). This latter result is consistent with a recent report indicating that angiotensinogen mRNA levels in humans is higher in the visceral than subcutaneous fat depot. Others that showed high differential expression were thrombospondin 1, a secreted protein that potentiates VEGF action; mevalonate kinase, an enzyme involved in biosynthesis of cholesterol and isoprenoids; the prolactin receptor and a novel transmembrane 4 superfamily member.

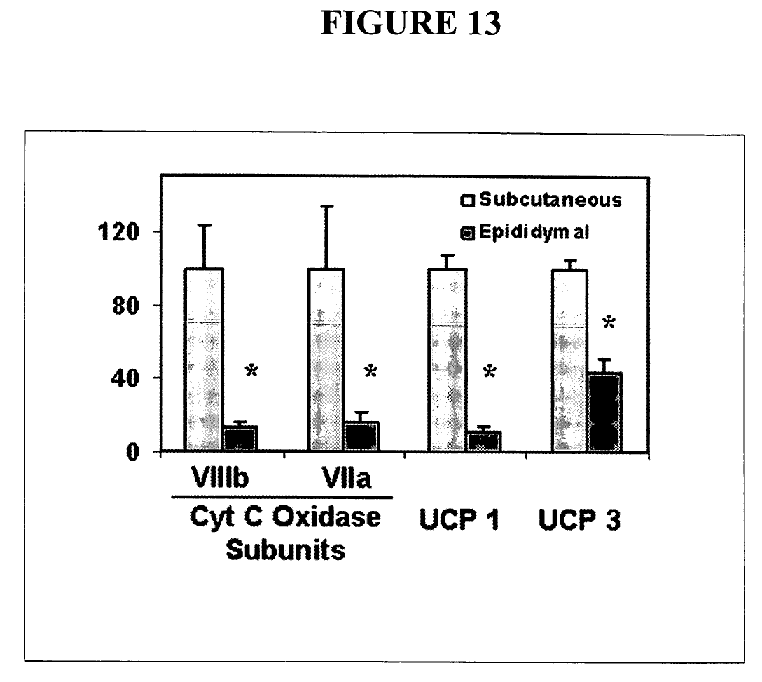

In addition, there were substantial changes in a number of genes of oxidative phosphorylation. Expression of multiple genes in oxidative phosphorylation pathways is reduced 2- to 9-fold in epididymal as compared with subcutaneous adipocytes isolated from C57BL/6J mice, including 5 genes in complex IV of electron transport, UCP 1, and UCP 3 (selected genes, relative expression normalized to subcutaneous values, FIG. 13).

Even more striking were the 248 genes that were more highly expressed in subcutaneous adipocytes, especially the finding that 4 members in one family of genes were 50-300 times more highly expressed in subcutaneous adipocytes. These genes were members of the family of “major urinary proteins” (MUPs). MUPs have been thought to be secreted by the liver and various exocrine glands and filtered by the kidneys into the urine of adult mice and rats, where they serve as binders of natural odorant molecules that serve as pheromones. On the other hand, MUPs are clearly highly expressed in subcutaneous fat and share a common ancestry with a number of hydrophobic ligand-binding proteins, such as serum retinol-binding proteins and fatty acid binding proteins. We find that MUP1, 3, 4 and 5 are selectively expressed in subcutaneous as compared to epididymal adipocytes. Also in this list was UCP1, which had a level 9 times higher in subcutaneous than in epididymal fat, the GABA1 receptor, and two homeobox transcription factors, engrailed-1 and SHOX2 (Table 2)

TABLE 2
Differences in Gene Expression in Adipose Tissue Depots

| Name | Epi | SC | Epi/SC | p value |
|---|---|---|---|---|
| Cytochrome P450.1b1 | 1289 | 117 | 11.0 | 0.039 |
| Angiotensinogen | 16339 | 2185 | 7.5 | 0.007 |
| Thrombospondin 1 | 8011 | 1387 | 5.8 | 0.023 |
| EST | 8216 | 2154 | 3.8 | 0.007 |
| Transmembrane 4 | 561 | 148 | 3.8 | 0.015 |
| superfamily member 9 | | | | |
| Prolactin receptor | 2057 | 546 | 3.8 | 0.003 |
| Name | Epi | SC | SC/Epi | p value |
| MUP1 | 150 | 42639 | 282.4 | 0.004 |
| MUP3 | 397 | 18991 | 47.8 | 0.013 |
| MUP5 | 386 | 11204 | 29.0 | 0.005 |
| MUP4 | 339 | 7319 | 21.5 | 0.006 |
| Uncoupling protein.1 | 143 | 1326 | 9.2 | 0.011 |
| SHOX 2 | 282 | 2456 | 8.7 | 0.001 |

### Example 6

### Experimental Methods

Animals and Genotyping

IR (lox/lox) mice derived from 129Sv and C57B1/6 chimeras were created by homologous recombination using an insulin receptor gene targeting vector with loxP sites flanking exon 4 as previously described (Brüning et al., 1998). FVB mice carrying the aP2-Cre transgene were made by cloning a 1.4 kb SacI/SalI complementary DNA fragment encoding Cre recombinase, modified by inclusion of a nuclear localization sequence (NLS) and a consensus polyadenylation signal, immediately downstream of the 5.4 kb promoter/enhancer of fatty-acid-binding protein aP2 (Abel et al., 2001) (FIG. 1a). Adipose tissue or fat specific insulin receptor knockout mice (FIRKO) were derived by crossing double heterozygous IR (lox/+) with IR (lox/+) mice that also expressed Cre recombinase under the control of the aP2 promoter/enhancer [aP2-Cre-IR(lox/+)].

Animals were housed in virus-free facilities on a 12 hr light/dark cycle (0700 on-1900 off) and were fed a standard rodent chow (Mouse Diet 9F, PMI Nutrition International) and water ad libitum. All protocols for animal use and euthanasia were reviewed and approved by the Animal Care Committee of the Joslin Diabetes Center and were in accordance with NIH guidelines. Genotyping was performed by PCR using genomic DNA isolated from the tail tip as previously described (Brüning et al. 1998). The 5′ and 3′ primers for the Cre transgene were 5′-ATG TCC AAT TTA CTG ACC G-3′ and 5′-CGC CGC ATA ACC AGT GAA AC-3′ and for the IR lox gene were 5′-GAT GTG CAC CCC ATG TCT G-3′ and 5′-CTG AAT AGC TGA GAC CAC AG-3′. The assessment of insulin receptor recombination was performed with DNA from isolated adipocytes of each animal using a previously described PCR strategy (Kulkarni et al., 1999) (FIG. 1b) in which a 250 bp amplified product indicated an intact exon 4, a 220 bp product suggested the presence of Cre mediated recombination, and a 300 bp product represented insulin receptor genes with an intact exon 4 flanked by a loxP site (FIG. 1c).

Isolation of adipocytes, adipocyte size and glucose transport Animals were anesthetized with sodium amobarbital (Eli Lilly, 75 mg/kg), and periovarian or epididymal fat pads were removed. Adipocytes were isolated by collagenase (1 mg/ml) digestion. Separation of cells into different diameter fractions was achieved by filtering the adipocyte suspension through serial nylon mesh screens with pore sizes of 25, 75, 100, 150 and 400 μm (Etherton et al., 1981). Aliquots of adipocytes were fixed with osmic acid and counted in a Coulter counter (Cushman et al., 1978). Adipocyte mass was determined by dividing the lipid content of the cell suspension by the cell number (Cushman et al., 1978). For the determination of glucose transport, isolated adipocytes of different diameter fractions were stimulated with 100 nM insulin for 30 min than incubated for 30 min with 3 μM U-14C-glucose (Tozzo et al., 1997). Immediately after the incubation adipocytes were fixed with osmic acid, incubated for 48 hours at 37° (Etherton et al., 1977), and the radioactivity was quantitated after the cells had been decolorized.

Immunoprecipitations and Western Blot Analysis

Tissues were removed and homogenized as previously described (Michael et al., 2000). Immunoprecipitations and Western blot analyses were performed on homogenates from isolated adipocytes. For each determination, cells were pooled from four WT, IR (lox/lox), and aP2-Cre mice or eight FIRKO mice, respectively. FIRKO mice were used only after confirmation of efficient insulin receptor knockout by IR rearrangement PCR (see above). For the analysis of insulin receptor expression, protein extracts from white and brown adipose tissue, liver, skeletal muscle, heart, and brain (FIG. 1d) were subjected to immunoprecipitation using insulin receptor specific antisera followed by Western blot analysis with the same antibody (Araki et al., 1994). At least three blots of samples from four (controls) to eight animals (FIRKO) of each genotype were scanned using a Molecular Dynamics Storm PhosphorImager, and signals were quantified using ImageQuant version 4.0 software. Statistical analysis of the data was performed using a two-tailed unpaired t-test, and significance was rejected at p>0.05.

Analytical Procedures

Blood glucose values were determined using whole venous blood and an automatic glucose monitor (Glucometer, Bayer). Serum insulin levels were measured by ELISA using mouse insulin as a standard (Crystal Chem, Chicago, Ill.). Serum triglyceride levels were measured in fasted animals by colorimetric enzyme assay using the GPO-Trinder Assay (Sigma). Serum free fatty acid levels were analyzed on fasted animals using the NEFA-Kit-U (Wako Chemicals GmBH, Neuss, Germany) with oleic acid as a standard.

Glucose tolerance tests were performed on animals that had been fasted overnight for 16 hours, whereas insulin tolerance tests were performed in the fed state at 1400 hr. Animals were injected with either 2 g/kg body weight of glucose or 1 U/kg body weight of human regular insulin (Eli Lilly) into the peritoneal cavity. Glucose levels were measured from blood collected from the tail immediately before and 15, 30, 60, and 120 min after the injection. Plasma leptin was measured using the rat leptin RIA kit (Linco Research, St Louis, Mo.). Body lipid (triglyceride) content of six mice from each genotype was determined by enzymatic measurement of glycerol after digestion of the carcass in 3 M KOH for 7 days at 60° C. (Sigma).

Goldthioglucose Treatment

At least eight 4 weeks old male mice from each genotype were injected intraperitoneally with a single dose of 0.5 mg/g body weight GTG (Fluka) in normal saline or normal saline (control animals). Food intake of 4 weeks old male FIRKO and controls littermates was determined daily over a week before and 12 weeks after goldthioglucose (GTG) or saline injection. Body weight was determined at least once per week and 12 weeks after the GTG injection glucose and insulin tolerance tests were performed in addition to metabolite measurements.

Histology

Tissues were fixed in 10% buffered formalin and imbedded in paraffin. Multiple sections (separated by 70-80 μm each) were obtained from gonadal fat pads and analyzed systematically with respect to adipocyte size and number. Staining of the sections was performed with hematoxylin/eosin. For each genotype and gender at least 10 fields (representing approximately 100 adipocytes) per slide were analyzed. Images were acquired using BX60 microscope (Olympus, N.Y.) and a HV-C20 TV camera (Hitachi, Japan) and were analyzed using Image-Pro Plus 4.0 software.

Statistical Analyses

All values are expressed as mean±SEM unless otherwise indicated. Statistical analyses were carried out using two-tailed Student's unpaired t-test and among more than two groups by analysis of variance (ANOVA). Significance was rejected at p≧0.05. Regression analyses were performed to evaluate the relation between leptin serum levels, body weight, and fat pad mass.

### REFERENCES

Abel et al. (2001). Adipose-selective targeting of the GLUT4 gene impaires insulin action in muscle and liver. Nature 409, 729-733
Araki et al. (1994). Alternative pathway of insulin signaling in mice with targeted disruption of the IRS-1 gene. Nature 372, 186-190
Bradley et al. (1999). Regulation of ob gene expression and leptin secretion by insulin and dexamethasone in rat adipocytes. Diabetes 48, 272-278
Brüning et al. (2000). Role of brain insulin receptor in control of body weight and reproduction. Science 289, 2122-2125
Brüning et al. (1998). A muscle-specific insulin receptor knockout exhibits features of the metabolic syndrome of NIDDM without altering glucose tolerance. Mol. Cell 2, 559-569
Cushman and Salans (1978). Determinations of adipose cell size and number in suspension of isolated rat and human adipose cells. J. Lipid Res. 19, 269-273
D'Adamo et al. (1998). Increased OB gene expression leads to elevated plasma leptin concentrations in patients with chronic primary hyperinsulinemia. Diabetes 47, 1625-1629
Debons et al. (1977). Gold thioglucose obesity syndrome. Fed. Proc. 36, 143-147
DeFronzo (1997). Pathogenesis of type 2 diabetes: metabolic and molecular implications for identifying diabetes genes. Diabetes Rev. 5, 177-269
Etherton et al. (1981). Effects of cell size and animal age on glucose metabolism in pig adipose tissue. J. Lipid Res. 22, 72-80
Etherton et al. (1977). Improved techniques for studies of adipocyte cellularity and metabolism. J. Lipid Res. 18, 552-557
Foley et al. (1980). Insulin binding and hexose transport in rat adipocytes. Diabetologia 19, 234-241
Glasow et al. (2001). Expression of leptin (Ob) and leptin receptor (Ob-R) in human fibroblasts: regulation of leptin secretion by insulin. J. Clin. Endocrinol. Metab. 86, 4472
Gu et al. (1994). Deletion of a DNA polymerase beta gene segment in T cells using cell type-specific gene targeting. Science 265, 103-106
Guerra et al. (2001). Brown adipose tissue-specific insulin receptor knockout shows phenotype without insulin resistance. J. Clin. Invest. 108, 1205-1213
Hajduch et al. (1992). Expression of glucose transporters (GLUT 1 and GLUT 4) in primary culturted rat adipocytes: differential evolution with time and chronic insulin effect. J. Cell. Biochem. 49, 251-258
James et al. (1985). Time dependence of insulin action in muscle and adipose tissue in the rat in vivo. An increasing response in adipose tissue with time. Diabetes 34, 1049-1054
Jansson et al. (1994). Lactate release from the subcutaneous tissue in lean and obese men. J. Clin. Invest. 93, 240-246
Joffe et al. (2001). From lipodystrophy syndromes to diabetes mellitus. Lancet 357, 1379-81
Kahn (1994). Insulin action, diabetogenes, and the cause of type II diabetes (Banting Lecture). Diabetes 43, 1066-1084
Kahn et al. (2000). Knockout mice challenge our concepts of glucose homeostasis and the pathogenesis of diabetes mellitus. J. Pediatric. Endo. Metab. 13, 1377-1384
Kashiwagi et al. (1983). In vitro insulin resistance of human adipocytes isolated from subjects with non-insulin-dependent diabetes mellitus. J Clin Invest 72, 1246-1254
Kopelman (2000). Obesity as a medical problem. Nature 404, 635-643
Kulkami et al. (1999). Tissue-specific knockout of the insulin receptor in pancreatic β cells creates an insulin secretory defect similar to that in type 2 diabetes. Cell 96, 329-339
Livingston et al. (1978). Insulin dependent regulation of the insulin-sensitivity of adipocytes. Nature 273, 394-396
Maffei et al. (1995). Leptin levels in human and rodent: measurement of plasma leptin and ob RNA in obese and weight-reduced subjects. Nature Medicine 1, 1155-61
Martin et al. (1992). Role of glucose and insulin resistance in development of type II diabetes mellitus: results of a 25-year follow-up study. Lancet 340, 925-929
Michael et al. (2000). Loss of insulin signaling in hepatocytes leads to severe insulin resistance and progressive hepatic dysfunction. Mol. Cell 6, 87-97
Mueller et al. (1998). Evidence that glucose metabolism regulates leptin secretion from cultured rat adipocytes. Endocrinology 139, 551-558
Postic et al. (1999). Dual roles for glucokinase in glucose homeostasis as determined by liver and pancreatic beta cell-specific gene knock-outs using Cre recombinase. J. Biol. Chem. 274, 305-15
Ross et al. (1990). A fat-specific enhancer is the primary determinant of gene expression for adipocyte P2 in vivo. Proc. Natl. Acad. Sci. USA 87, 9590-9594
Sakoda et al. (2000). Dexamethasone-induced insulin resistance in 3T3-L1 adipocytes is due to inhibition of glucose transport rather than insulin signal transduction. Diabetes 49, 1700-1708
Szanto and Kahn (2000). Selective interaction between leptin and insulin signaling pathways in a hepatic cell line. Proc. Natl. Acad. Sci. USA 97, 2355-2360
Tozzo et al. (1997). Amelioration of insulin resistance in streptozotocin diabetic mice by transgenic overexpression of GLUT4 driven by an adipose-specific promoter. Endocrinology 138, 1604-1611
Zambrowicz et al. (1997). Disruption of overlapping transcripts in the ROSA beta geo 26 gene trap strain leads to widespread expression of beta-galactosidase in mouse embryos and hematopoietic cells. Proc. Natl. Acad. Sci. USA 94, 3789-3794
Zhao et al. (2000). Leptin induces insulin-like signaling that antagonizes cAMP elevation by glucagon in hepatocytyes. J. Biol. Chem. 14, 11348-11354
Zisman et al. (2000). Targeted disruption of the glucose transporter 4 selectively in muscle causes insulin resistance and glucose intolerance. Nature Medicine 6, 924-928

1. An isolated population of adipose cells that can exhibit decreased expression of one or more of: fatty acid synthase (FAS), SREBP-1c, C/EBPα, β3 adrenergic receptor, and fatty acid transport protein 1 (FATP1), compared to a reference.

2. The population of claim 1, wherein the decreased expression is exhibited in response to inhibition of insulin signaling.

3. The population of claim 1, wherein the population has an average diameter of less than 75 μm.

4. The population of claim 1, wherein the population has an average diameter of less than 50 μm.

5. The population of claim 1, wherein the population has an average diameter of less than 25 μm.

6. The population of claim 1, wherein the population has decreased basal lipogenesis activity.

7. The population of claim 1, wherein the population has decreased isoproterenol induced lipolysis activity.

8. An isolated population of white adipose cells having an average diameter of less than 80% of the median diameter of the white adipose cells before isolation.

9. The population of claim 8, wherein the isolated population has an average cell diameter of less than 75 μm.

10. The population of claim 8, wherein the isolated population has an average cell diameter of less than 50 μm.

11. The population of claim 8, wherein the isolated population has an average cell diameter of less than 25 μm.

12. The population of claim 8, wherein the population has decreased transcription of one or more of: fatty acid synthase (FAS), SREBP-1c, C/EBPα, β3 adrenergic receptor, and fatty acid transport protein 1 (FATP1), in response to inhibition of insulin signaling

13. The population of claim 8, wherein the population has decreased basal lipogenesis activity.

14. The population of claim 8, wherein the population has decreased isoproterenol induced lipolysis activity.

15. The population of claim 8, wherein the population has decreased levels or activity of one or more of: fatty acid synthase (FAS), fatty acid transport protein aP2 (FATP aP2), carnitine palmitoyltransferase 2, non-muscle myosin form A, acetyl CoA-dehydrogenase, citrate synthase and cytochrome C.

16. A population of isolated white adipocyte cells which, in response to inhibition of insulin signaling, exhibit an average diameter of at least 120% of the median diameter of the white adipose cells before isolation.

17. The population of claim 16, wherein the population has an average diameter of at least 100 μm.

18. The population of claim 16, wherein the population has an average diameter of at least 125 μm.

19. The population of claim 16, wherein the population has an average diameter of at least 150 μm.

20. The population of claim 16, wherein the cells have increased basal lipogenesis and/or increased basal lipolysis activity.

21. A method of screening a treatment or compound for modulating weight or fat composition, the method comprising:
(a) providing or obtaining the adipose cell population of any one of the preceding claims;
(b) contacting the population with a test treatment or compound; and
(c) evaluating a parameter of fat cell size, function or gene expression in the population,
wherein a test compound that modulates a parameter of fat cell size, function or gene expression in the population is identified as a treatment or compound for modulating weight or fat composition.

22. The method of claim 21, wherein expression, levels or activity of FAS, SREBP-1c, C/EBPα, β3 adrenergic receptor, FATP 1, FATP aP2, carnitine palmitoyltransferase 2, non-muscle myosin form A, acetyl CoA-dehydrogenase, citrate synthase, cytochrome C, vimentin, EH domain containing protein 2, elongation factor 2, GRP 78, transketolase, succinyl CoA transferase, activating transcription factor 3, TGFβ, PAI 1, annexin 2, annexin A6, 47 kDa heat shock protein, PDGF receptor, lumican or CSF3 is evaluated.

23. The method of claim 21, wherein glucose uptake, lipogenesis and/or lipolysis is evaluated.

24. The method of claim 21, wherein cell diameter is evaluated.

25. The method of claim 22, further comprising administering the test treatment or compound to an experimental animal.

26. A method of identifying or selecting an adipocyte, the method comprising:
providing a test adipocyte; and
evaluating gene expression, level or activity of one or more of: fatty acid synthase (FAS), SREBP-1c, C/EBPα, β3 adrenergic receptor, and fatty acid transport protein 1 (FATP1), fatty acid transport protein aP2 (FATP aP2), carnitine palmitoyltransferase 2, non-muscle myosin form A, acetyl CoA-dehydrogenase, citrate synthase and cytochrome C in the test adipocyte,
wherein the test adipocyte is identified or selected if the gene expression, level or activity is decreased compared to a reference.

27. The method of claim 26, wherein the method further comprises a step of isolating the selected adipocyte.

28. The method of claim 26, wherein the method comprises a step of inhibiting insulin signaling in the adipocyte.

29. The method of claim 28, wherein inhibiting insulin signaling in the adipocyte comprises inhibiting an insulin receptor.

30. The method of claim 26, wherein the gene expression or level or activity is decreased in response to inhibition of insulin signaling.

31. The method of claim 26, further comprising the step of evaluating glucose uptake, lipogenesis or lipolysis in the identified adipocyte.

32. A method of evaluating a subject's predisposition or risk of obesity or an obesity related disorder, the method comprising:
evaluating the subject for the presence, amount or ratio of TG-S adipocytes and/or TG-L adipocytes,
wherein a decreased presence, amount or ratio of TG-S adipocytes, compared to a reference value, indicates risk.

33. The method of claim 32, wherein the evaluation is performed on a fat tissue sample or an adipose cell sample from the subject.

34. The method of claim 32, wherein a ratio of TG-S to TG-L adipocytes is determined.

35. The method of claim 33, wherein the sample is contacted with an agent that decreases or inhibits insulin signaling.

36. The method of claim 33, wherein the sample is a subcutaneous fat tissue or adipose cell sample.

37. A method of modulating adipocyte differentiation, size or function, the method comprising:
providing, obtaining or identifying an adipocyte; and
modulating in the adipocyte the expression, levels or activity of one or more of: fatty acid synthase (FAS), SREBP-1c, C/EBPα, β3 adrenergic receptor, fatty acid transport protein 1 (FATP 1), fatty acid transport protein aP2 (FATP aP2), carnitine palmitoyltransferase 2, non-muscle myosin form A, acetyl CoA-dehydrogenase, citrate synthase, cytochrome C, vimentin, EH domain containing protein 2, elongation factor 2, glucose regulated protein 78 (GRP 78), transketolase, succinyl CoA transferase, activating transcription factor 3, TGFβ, PAI 1, annexin 2, annexin A6, 47 kDa heat shock protein, PDGF receptor, lumican and colony stimulating factor 3,
thereby modulating adipocyte differentiation, size or function.

38. The method of claim 37, wherein the expression, levels or activity is modulated in vitro.

39. The method of claim 37, wherein the expression, levels or activity is modulated in vivo.

40. The method of claim 37, wherein FAS, SREBP-1c, C/EBPα, β3 adrenergic receptor, or FATP 1 expression or activity is decreased.

41. The method of claim 37, wherein the adipocyte is from a subject with unwanted weight or fat.

42. The method of claim 37, further comprising inhibiting insulin signaling in the adipocyte.

43. The method of claim 37, wherein expression, levels or activity is modulated in vitro and the modulated adipocyte is transplanted into a subject.

44. A method of identifying a subject having, or at risk for, diabetes or a diabetes related condition (atherosclerosis, dyslipidemia, hypertension) the method comprising evaluating a fat cell or tissue of the subject for expression of one or more of: cytochrome p450 1b1, 17-beta-estradiol, angiotensinogen, thrombospondin 1, mevalonate kinase, and prolactin receptor.

45. The method of claim 44, wherein the fat cell or tissue is derived from a visceral depot.

46. The method of claim 44, wherein the subject is a human.

47. The method of claim 44, wherein the fat cell or tissue is evaluated in vitro.

48. The method of claim 44, wherein the fat cell or tissue is evaluated in vivo.

49. A method of identifying a subject having, or at risk for, diabetes or a diabetes related condition (atherosclerosis, dyslipidemia, hypertension) the method comprising evaluating a fat cell or tissue of the subject for expression of one or more protein in the mitochondrial oxidative phosphorylation pathway.

50. The method of claim 49, wherein the protein is selected from the group consisting of: cytochrome C oxidase subunit VIIIb, cytochrome C oxidase subunit VIIa, UCP1 and UCP3.

51. The method of claim 50, wherein the fat cell or tissue is derived from a visceral depot.

52. The method of claim 51, wherein the subject is a human.

53. The method of claim 52, wherein the fat cell is evaluated in vitro.

54. The method of claim 53, wherein the fat cell or tissue is evaluated in vivo.

55. A method of treating a subject for diabetes or a diabetes related disorder, the method comprising modulating one or more of: cytochrome p450 1b1, 17-beta-estradiol, angiotensinogen, thrombospondin 1, mevalonate kinase, prolactin receptor, and a protein in the mitochondrial oxidative phosphorylation pathway, in a fat cell or tissue of the subject.

56. The method of claim 55, wherein modulating is increasing.


SEQUENCE LISTING (4 sequences)

SEQ ID NO: 1 - DNA, 19 bp, Artificial Sequence
  Primer
atgtccaatttactgaccg

SEQ ID NO: 2 - DNA, 20 bp, Artificial Sequence
  Primer
cgccgcataaccagtgaaac

SEQ ID NO: 3 - DNA, 19 bp, Artificial Sequence
  Primer
gatgtgcaccccatgtctg

SEQ ID NO: 4 - DNA, 20 bp, Artificial Sequence
  Primer
ctgaatagctgagaccacag